(19) United States
(12) Patent Application Publication
(10) Pub. No.: US 2018/0023114 A1
(43) Pub. Date: Jan. 25, 2018

(54) Labile Linkers for Biomarker Detection

(71) Applicant: Two Pore Guys, Inc., Santa Cruz, CA (US)

(72) Inventors: Trevor J. Morin, Santa Cruz, CA (US); William B. Dunbar, Santa Cruz, CA (US); Daniel A. Heller, Santa Cruz, CA (US); Tyler Shropshire, Santa Cruz, CA (US)

(21) Appl. No.: 15/547,439

(22) PCT Filed: Feb. 2, 2016
(86) PCT No.: PCT/US2016/016235

§ 371 Date: Jul. 28, 2017

Related U.S. Application Data
(60) Provisional application No. 62/111,073, filed on Feb. 2, 2015.

(51) Int. Cl. C12Q 1/37; C12Q 1/14; G01N 33/487; C12Q 1/34

(52) U.S. Cl. CPC C12Q 1/37; C12Q 1/34; C12Q 1/14; G01N 33/48721

(57) ABSTRACT

Disclosed herein are methods and compositions for electronic detection and/or quantification of enzymes or enzymatic activity in a sample using a pore system.

### CROSS-REFERENCE TO RELATED APPLICATION

This application claims the benefit of U.S. Provisional Patent Application No. 62/111,073, filed Feb. 2, 2016, the disclosure of which is incorporated herein by reference.

### BACKGROUND

Enzymatic activity present in a sample can indicate the presence of toxins, a disorder, or other condition of an organism. For example, proteases are critically important molecules found in humans that regulate a wide variety of normal human physiological processes including wound healing, cell signaling, and apoptosis. Because of their critical role within the human body, abnormal protease activity has been associated with a number of disease states including, but not limited to, rheumatoid arthritis, Alzheimer's disease, cardiovascular disease and a wide range of malignancies. Prostate specific antigen (PSA) is one example of a valuable diagnostic protease that is the gold standard in diagnosing and monitoring prostate cancer in males. Proteases are found in nearly all human fluids and tissue, and their activity levels can signal the presence of a condition.

Although multiple strategies exist to determine the presence of an enzyme in a sample, often, the activity of the enzyme in question is more important than the presence or absence of the enzyme itself. Strategies do exist to assess the level of enzymatic activity present in a sample, but these techniques are often costly, require significant time investment and device infrastructure, and/or are difficult to use or non-portable. What is needed therefore, is a method of determining enzymatic activity in a solution that is fast, discriminates active enzymes from those that are merely present and non-active, is label free, and/or can be done on a purified or non-purified sample.

### SUMMARY

Various aspects disclosed herein may fulfill one or more of the above-mentioned needs. The systems and methods described herein each have several aspects, no single one of which is solely responsible for its desirable attributes. Without limiting the scope of this disclosure as expressed by the claims that follow, the more prominent features will now be discussed briefly. After considering this discussion, and particularly after reading the section entitled “Detailed Description,” one will understand how the sample features described herein provide for improved systems and methods.

In some embodiments, provided herein are methods of detecting the presence or absence of a target molecule or condition in a sample by detecting cleavage of a labile linker (e.g., a cleavable linker) by the target molecule or condition using a nanopore device to identify the products of the cleavage. In some embodiments, the target molecule is an enzyme, and the methods described herein detect the presence or absence of active target enzymes in the sample.

In certain preferred embodiments, the polymer scaffold is dsDNA. In certain preferred embodiments, the fusion is bound directly and covalently to the dsDNA, and the payload is bound directly and non-covalently to the fusion.

In some embodiments, prior to cleavage of the cleavable linker by an enzyme, the scaffold/fusion/payload provides a unique and detectable current upon translocation through the nanopore. In some embodiments, after cleavage of the cleavable linker by an enzyme, the scaffold (or scaffold plus remaining components of the fusion) and payload (or payload plus remaining components of the fusion) are no longer bound, and each provides a unique and detectable current upon translocation through the nanopore, which are distinct from the scaffold/fusion/payload complex.

In certain embodiments, the fusion molecule comprises PNA bound to the DNA scaffold, and the cleavable linker tethered covalently to the PNA by a connector. In certain embodiments, the payload is a PEG that is bound to the cleavable linker. In certain embodiments, the size, shape, and or charge of the payload may be modified to increase resolution based on current impedance in a pore of a specific shape or size, to provide improved discrimination between scaffold/fusion/payload complex and scaffold and payload.

In certain embodiments, the polymer scaffold is dsDNA with one or more sequence sites comprising a cleavable domain that is cleavable by one or more target endonucleases. In certain embodiments, the polymer scaffold is linear dsDNA prior to cleavage. In certain embodiments, the polymer scaffold is circularized dsDNA prior to cleavage.

Also provided herein are methods of analyzing data from a nanopore device to quantitate the presence of the target molecule or condition suspected to be present in a sample. In certain preferred embodiments, a numerical confidence value to detection is assigned. In certain preferred embodiments, the concentration of the target is estimated by applying mathematical tools to repeated experiments that vary concentrations of one or more of the fusion, scaffold, payload, and/or target molecules.

In some embodiments, provided herein is a method of detecting the presence or absence of a target molecule suspected to be present in a sample, comprising: contacting the sample with a fusion molecule comprising a cleavable linker, wherein the cleavable linker is specifically cleaved in the presence of the target molecule; loading the sample into a device comprising a nanopore, wherein the nanopore separates an interior space of the device into two volumes; configuring the device to pass a polymer scaffold through the nanopore, wherein a first portion of the fusion molecule is bound to the polymer scaffold, wherein a second portion of the fusion molecule is bound to a payload molecule, and wherein the device comprises a sensor configured to identify objects passing through the nanopore; and determining with the sensor whether the cleavable linker has been cleaved, thereby detecting the presence or absence of the target molecule in the sample.

In some embodiments, contacting the sample with the fusion molecule is performed prior to loading the sample into the device. In some embodiments, loading the sample into the device is performed prior to contacting the sample with the fusion molecule.

In some embodiments, the fusion molecule comprises a polymer scaffold binding domain. In some embodiments, the method of detecting the presence or absence of a target molecule further comprises contacting the sample with a polymer scaffold. In some embodiments, the method of detecting the presence or absence of a target molecule further comprises binding the polymer scaffold to the polymer scaffold binding domain. In some embodiments, the polymer scaffold is bound to the polymer scaffold binding domain via a covalent bond, a hydrogen bond, an ionic bond, a van der Waals force, a hydrophobic interaction, a cation-pi interaction, a planar stacking interaction, or a metallic bond. In some embodiments, the polymer scaffold binding domain comprises an azide group. In some embodiments, the polymer scaffold binding domain comprises a molecule selected from the group consisting of: DNA, RNA, PNA, polypeptide, a cholesterol/DNA hybrid, and a DNA/RNA hybrid. In some embodiments, the polymer scaffold binding domain comprises a molecule selected from the group consisting of: a locked nucleic acid (LNA), a bridged nucleic acid (BNA), a transcription activator-like effector nuclease (TALEN), a clustered regularly interspaced short palindromic repeat (CRISPR), an aptamer, a DNA binding protein, and an antibody fragment. In some embodiments, the DNA binding protein comprises a zinc finger protein. In some embodiments, the antibody fragment comprises a fragment antigen-binding (Fab) fragment. In some embodiments, the polymer scaffold binding domain comprises a chemical modification.

In some embodiments, the fusion molecule comprises a payload molecule binding domain. In some embodiments, the method of detecting the presence or absence of a target molecule further comprises contacting the sample with a payload molecule.

In some embodiments, the method of detecting the presence or absence of a target molecule further comprises binding the payload molecule to the payload molecule binding domain. In some embodiments, the payload molecule binds to the payload molecule binding domain via a covalent bond, a hydrogen bond, an ionic bond, a van der Waals force, a hydrophobic interaction, a cation-pi interaction, a planar stacking interaction, or a metallic bond. In some embodiments, the payload molecule binding domain comprises DBCO.

In some embodiments, the fusion molecule comprises a polymer scaffold binding domain and a payload molecule binding domain. In some embodiments, the first portion of the fusion molecule is bound directly or indirectly to the polymer scaffold via a covalent bond, a hydrogen bond, an ionic bond, a van der Waals force, a hydrophobic interaction, a cation-pi interaction, a planar stacking interaction, or a metallic bond. In some embodiments, the second portion of the fusion molecule is bound directly or indirectly to the payload molecule via a covalent bond, a hydrogen bond, an ionic bond, a van der Waals force, a hydrophobic interaction, a cation-pi interaction, a planar stacking interaction, or a metallic bond.

In some embodiments, the payload molecule or the polymer scaffold is bound to the fusion molecule via direct covalent tethering. In some embodiments, the fusion molecule comprises a connector for direct covalent tethering of the polymer scaffold or the fusion molecule to the cleavable linker. In some embodiments, the polymer scaffold comprises the fusion molecule. In some embodiments, detection of the presence or absence of the target molecule in the sample comprises determining with a sensor whether the polymer scaffold is bound to the payload molecule via the fusion molecule. In some embodiments, the sensor detects an electrical signal in the nanopore. In some embodiments, the electrical signal is an electrical current.

In some embodiments, the target molecule is a hydrolase or lyase. In some embodiments, the cleavable linker comprises a molecule selected from the group consisting of: a deoxyribonucleic acid (DNA), a ribonucleic acid (RNA), and a polypeptide. In some embodiments, the cleavable linker is selected from the group consisting of: an azo compound, a disulfide bridge, a sulfone, an ethylene glycolyl disuccinate, a hydrazone, an acetal, an imine, a vinyl ether, a vicinal diol, and a picolinate ester. In some embodiments, the target molecule specifically cleaves a bond in the cleavable linker selected from the group consisting of: a carbon-oxygen bond, a carbon-sulfur bond, a carbon-nitrogen bond, and a carbon-carbon bond.

In some embodiments, the polymer scaffold comprises a molecule selected from the group consisting of: a deoxyribonucleic acid (DNA), a dendrimer, a peptide nucleic acid (PNA), a ribonucleic acid (RNA), a polypeptide, a nanorod, a nanotube, a cholesterol/DNA hybrid, and a DNA/RNA hybrid. In some embodiments, the payload molecule comprises a molecule selected from the group consisting of: a dendrimer, a double stranded DNA, a single stranded DNA, a DNA aptamer, a fluorophore, a protein, a polypeptide, a nanobead, a nanorod, a nanotube, a fullerene, a PEG molecule, a liposome, and a cholesterol-DNA hybrid. In some embodiments, the fusion molecule comprises two or more cleavable linkers.

In some embodiments, the sensor comprises an electrode pair, wherein the electrode pair applies a voltage differential between the two volumes and detects current flow through the nanopore. In some embodiments, the device comprises at least two nanopores in series, wherein the polymer scaffold is simultaneously captured and detected in the at least two nanopores. In some embodiments, the translocation of the polymer scaffold is controlled by applying a unique voltage across each of the nanopores.

Also provided herein is a method of detecting the presence or absence of a target molecule or condition suspected to be present in a sample, the method comprising: contacting the sample with a fusion molecule comprising a cleavable linker, wherein the cleavable linker is specifically cleaved in the presence of the target molecule or condition; loading the sample into a device comprising a nanopore, wherein the nanopore separates an interior space of the device into two volumes; configuring the device to pass a polymer scaffold through the nanopore, wherein a first portion of the fusion molecule is bound to the polymer scaffold, wherein a second portion of the fusion molecule is bound to a payload molecule, and wherein the device comprises a sensor configured to identify objects passing through the nanopore; and determining with the sensor whether the cleavable linker has been cleaved, thereby detecting the presence or absence of the target molecule or condition in the sample.

In some embodiments, provided herein is a method for detecting the presence or absence of a target molecule or condition suspected to be present in a sample, the method comprising: contacting the sample with a fusion molecule, a polymer scaffold, and a payload molecule, the fusion molecule comprising a cleavable linker, wherein the target molecule specifically cleaves the cleavable linker, a polymer scaffold binding domain, and a payload molecule binding domain; loading the fusion molecule, the polymer scaffold, the payload molecule, and the sample into a device comprising a nanopore, wherein the nanopore separates an interior space of the device into two volumes; configuring the device to pass the polymer scaffold through the nanopore, wherein the device comprises a sensor configured to identify objects passing through the nanopore; and determining with the sensor whether the cleavable linker is bound to the payload molecule, thereby detecting the presence or absence of the target molecule or condition.

In some embodiments, the target molecule comprises a hydrolase or lyase. In some embodiments, the target molecule or condition photolytically cleaves the cleavable linker via exposure of the cleavable linker to light comprising a wavelength of 10 nm to 550 nm. In some embodiments, the cleavable linker sensitive to photolytic cleavage is selected from the group consisting of: an ortho-nitrobenzyl derivative and a phenacyl ester derivative. In some embodiments, the target molecule or condition chemically cleaves the cleavable linker via exposure of the cleavable linker to a reagent selected from the group consisting of: a nucleophilic reagent, a basic reagent, an electrophilic reagent, an acidic reagent, a reducing reagent, an oxidizing reagent, and an organometallic compound.

In some embodiments, at least one of the two volumes in the device comprises conditions allowing binding of the fusion molecule to the polymer scaffold and binding of the fusion molecule to the payload molecule. In some embodiments, the fusion molecule is bound to the polymer scaffold and the payload molecule prior to contacting the sample with the fusion molecule. In some embodiments, the fusion molecule is bound to the polymer scaffold and the payload molecule, prior to loading the fusion molecule into the device.

In some embodiments, one or more volumes within the device comprises conditions allowing the target molecule or the condition suspected to be present in the sample to cleave the cleavable linker. In some embodiments, contacting the sample with the fusion molecule is performed prior to loading the sample into the device. In some embodiments, loading the sample into the device is performed prior to contacting the sample with the fusion molecule.

In some embodiments, the polymer scaffold comprises a molecule selected from the group consisting of: deoxyribonucleic acid (DNA), a dendrimer, a peptide nucleic acid (PNA), a ribonucleic acid (RNA), a polypeptide, a nanorod, a nanotube, a cholesterol/DNA hybrid, and a DNA/RNA hybrid.

In some embodiments, the cleavable linker comprises a molecule selected from the group consisting of: a deoxyribonucleic acid (DNA), a ribonucleic acid (RNA), and a polypeptide. In some embodiments, the cleavable linker is selected from the group consisting of: an azo compound, a disulfide bridge, a sulfone, an ethylene glycolyl disuccinate, a hydrazone, an acetal, an imine, a vinyl ether, a vicinal diol, and a picolinate ester.

In some embodiments, the target molecule or condition specifically cleaves a bond in the cleavable linker selected from the group consisting of: a carbon-oxygen bond, a carbon-sulfur bond, a carbon-nitrogen bond, and a carbon-carbon bond.

In some embodiments, the payload molecule comprises a molecule selected from the group consisting of: a dendrimer, a double stranded DNA, a single stranded DNA, a DNA aptamer, a fluorophore, a protein, a polypeptide, a nanobead, a nanorod, a nanotube, a fullerene, a PEG molecule, a liposome, and a cholesterol-DNA hybrid.

In some embodiments, the polymer scaffold and the fusion molecule are bound via a covalent bond, a hydrogen bond, an ionic bond, a van der Waals force, a hydrophobic interaction, a cation-pi interaction, a planar stacking interaction, or a metallic bond. In some embodiments, the scaffold and the fusion molecule are bound via direct covalent tethering. In some embodiments, the fusion molecule comprises a connector for direct covalent tethering to the polymer scaffold, wherein the connector is bound to the cleavable linker. In some embodiments, the connector comprises polyethylene glycol. In some embodiments, the fusion molecule comprises a polymer scaffold binding domain comprising a molecule selected from the group consisting of: DNA, RNA, PNA, polypeptide, a cholesterol/DNA hybrid, and a DNA/RNA hybrid.

In some embodiments, the fusion molecule comprises a molecule selected from the group consisting of: a locked nucleic acid (LNA), a bridged nucleic acid (BNA), a transcription activator-like effector nuclease (TALEN), a clustered regularly interspaced short palindromic repeat (CRISPR), an aptamer, a DNA binding protein, and an antibody fragment. In some embodiments, the DNA binding protein comprises a zinc finger protein. In some embodiments, the antibody fragment comprises a fragment antigen-binding (Fab) fragment. In some embodiments, the fusion molecule comprises a chemical modification.

In some embodiments, the cleavable linker and the payload molecule are bound directly or indirectly via a covalent bond, a hydrogen bond, an ionic bond, a van der Waals force, a hydrophobic interaction, a cation-pi interaction, a planar stacking interaction, or a metallic bond. In some embodiments, the fusion molecule comprises two or more cleavable linkers.

In some embodiments, the sensor comprises an electrode pair, wherein the electrode pair applies a voltage differential between the two volumes and detects current flow through the nanopore.

Also provided herein is a method for detecting a target molecule or condition suspected to be present in a sample, the method comprising: contacting the sample with a polymer scaffold, wherein the scaffold comprises a cleavable domain, wherein the cleavable domain is specifically cleaved in the presence of the target molecule; loading the polymer scaffold and the sample into a device comprising a nanopore, wherein the nanopore separates an interior space of the device into two volumes; configuring the device to pass the polymer scaffold through the nanopore, wherein the device comprises a sensor configured to identify objects passing through the nanopore; and determining with the sensor whether the cleavable domain has been cleaved, thereby detecting the presence or absence of the target molecule or condition in the sample.

In some embodiments, the polymer scaffold comprises a molecule selected from the group consisting of: a deoxyribonucleic acid (DNA), a dendrimer, a peptide nucleic acid (PNA), a ribonucleic acid (RNA), a polypeptide, a nanorod, a nanotube, a cholesterol/DNA hybrid, and a DNA/RNA hybrid.

In some embodiments, the cleavable domain comprises a molecule selected from the group consisting of: a deoxyribonucleic acid (DNA), a ribonucleic acid (RNA), and a polypeptide. In some embodiments, the target molecule or condition specifically cleaves a bond of the cleavable domain selected from the group consisting of: a carbon-oxygen bond, a carbon-sulfur bond, a carbon-nitrogen bond, and a carbon-carbon bond.

In some embodiments, the cleavable domain is photolytically cleaved in the presence of the target molecule or condition, and wherein the cleavable domain comprises a molecule selected from the group consisting of: an ortho-nitrobenzyl derivative and a phenacyl ester derivative. In some embodiments, the cleavable domain is chemically cleaved in the presence of the target molecule or condition, and wherein the cleavable domain comprises a molecule selected from the group consisting of: an azo compound, a disulfide bridge, a sulfone, an ethylene glycolyl disuccinate, a hydrazone, an acetal, an imine, a vinyl ether, a vicinal diol, or a picolinate ester.

In some embodiments, the device comprises at least two nanopores in series, and wherein the polymer scaffold is simultaneously in the at least two nanopores during translocation.

In some embodiments, provided herein is a method of quantitating a target molecule or condition suspected to be present in a sample, the method comprising: contacting the sample with a fusion molecule, a polymer scaffold, and a payload molecule, the fusion molecule comprising a cleavable linker, wherein the cleavable linker is specifically cleaved in the presence of the target molecule or condition, a polymer scaffold binding domain, and a payload molecule binding domain; loading the fusion molecule, the polymer scaffold, the payload molecule, and the sample into a device comprising a nanopore, wherein the nanopore separates an interior space of the device into two volumes; configuring the device to pass the polymer scaffold through the nanopore, wherein the device comprises a sensor configured to identify objects passing through the nanopore; determining with the sensor whether the polymer scaffold is bound to the payload molecule, thereby detecting the presence or absence of target molecule; and estimating the concentration or activity of the target molecule or condition suspected to be present in a sample using measurements from the sensor.

In some embodiments, determination of the concentration or activity comprises assigning a numerical confidence value to detection of the target molecule or condition suspected to be present in the sample. In some embodiments, the steps of contacting the sample with the fusion molecule, loading the fusion molecule, the polymer scaffold, the payload molecule, and the sample into the device, configuring the device, and determining whether the polymer scaffold is bound to the payload molecule are repeated for varying concentrations or activity of one or more of the polymer scaffold, the fusion molecule, the payload molecule or the target molecule or condition in the sample.

Also provided herein is a method of quantitating a target molecule suspected to be present in a sample, the method comprising: contacting the sample with a fusion molecule comprising a cleavable linker, wherein the cleavable linker is specifically cleaved in the presence of the target molecule; loading the sample into a device comprising a nanopore, wherein the nanopore separates an interior space of the device into two volumes; configuring the device to pass a polymer scaffold through the nanopore, wherein a first portion of the fusion molecule is bound to the polymer scaffold, wherein a second portion of the fusion molecule is bound to a payload molecule, and wherein the device comprises a sensor configured to identify objects passing through the nanopore; determining with the sensor whether the cleavable linker has been cleaved, thereby detecting the presence or absence of the target molecule in the sample; and estimating the concentration of the target molecule or condition suspected to be present in a sample using measurements from the sensor.

In some embodiments, determination of the concentration comprises assigning a numerical confidence value to detection of the target molecule or condition suspected to be present in the sample. In some embodiments, the steps of contacting the sample with the fusion molecule, loading the sample into the device, configuring the device, and determining whether the cleavable linker has been cleaved are repeated for varying concentrations of one or more of the polymer scaffold, the fusion molecule, the payload molecule or the target molecule or condition in the sample.

Also provided herein is a kit comprising: a device comprising a nanopore, wherein the nanopore separates an interior space of the device into two volumes, and configuring the device to pass the nucleic acid through one or more pores, wherein the device comprises a sensor for each pore that is configured to identify objects passing through the nanopore; a fusion molecule comprising a cleavable linker, wherein the cleavable linker is specifically cleaved in the presence of a target molecule; a payload molecule; a polymer scaffold; and instructions for use to detect the presence or absence of the target molecule in a sample.

In some embodiments, the fusion molecule is bound to the payload molecule. In some embodiments, the fusion molecule is bound to the polymer scaffold.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention. Provided also as embodiments of this disclosure are data figures that illustrate features by exemplification only, and not limitation.

FIG. 1 depicts an embodiment of the fusion molecule with a cleavable linker, the fusion bound to a payload, and the fusion bound to a scaffold captured in a nanopore.

FIG. 2 depicts one method of using a scaffold/fusion/payload molecule to detect enzymatic activity using a nanopore system.

FIG. 3 depicts a method of using a linear scaffold molecule to detect endonuclease activity with a nanopore.

FIG. 4 depicts a method of using a circularized scaffold molecule to detect endonuclease activity with a nanopore.

FIG. 5 depicts a method of detection of multiplexed detection of endonuclease activity with a nanopore using a target molecule with multiple target sites.

FIGS. 6A, 6B, and 6C depicts a specific example of a cleavable linker susceptible to proteolytic degradation by matrix-metalloproteinase 9 (MMP9) that is included within a fusion molecule. The linker component of the fusion molecule is connected to a PEG-biotin payload (FIG. 6A), or a PEG-biotin-monostreptavidin payload that is larger in size (FIG. 6B). The fusion molecule contains an azide chemical group (N₃) that is capable of chemically coupling to the DNA scaffold molecule via “click” chemistry (FIG. 6C).

FIG. 7 illustrates the example of proteolytic degradation of a cleavable linker by MMP9. Upon incubation with a sample containing MMP9, the protease-sensitive construct is cleaved into two separate fragments.

FIG. 8 depicts idealized current profiles of three example molecules when passing through a nanopore whose impedance values indicate whether or not the cleavable linker has been proteolytically digested. The deeper and longer lasting current impedance profile of an intact DNA scaffold/fusion/payload shown in Panel A indicates the cleavable linker was not been degraded by MMP9. Briefer and/or shallower current impedance profiles are shown in Panels B and C for the two fragments following cleavage of the cleavable linker by MMP9, with the fragments being smaller than the full scaffold/fusion/payload complex and therefore impeding less current when each passes through a nanopore.

FIG. 9A depicts a specific example of a double-stranded DNA that comprises the scaffold molecule and a portion of the fusion molecule that contains a specific DNA sequence susceptible to cleavage by an endonuclease of interest. The fusion molecule also contains a dibenzocyclooctyne (DBCO) chemical handle for downstream conjugation to a payload molecule via copper-free “click” chemistry. In FIG. 9B, the DBCO handle is conjugated to a PEG-biotin payload.

FIG. 10 illustrates a specific example of the degradation of the cleavable domain sequence included in the linker region of the DNA by the endonuclease Eco81I. Upon incubation of the full scaffold/fusion molecule/payload construct with a sample containing Eco81I, the specific sequence recognized by the endonuclease in the cleavable domain is cleaved, resulting in two separate fragments.

FIG. 11 depicts idealized current profiles of three example molecules whose impedance values indicate whether or not the sequence (i.e., the cleavable domain) encoded in the fusion component of the DNA has been digested by an endonuclease. Panel A depicts an idealized current profile of an intact scaffold/fusion/payload when passing through a nanopore, with the large impedance of the full molecular construct indicating that the endonuclease has not cleaved the cleavable domain sequence. Panel B depicts an idealized current profile of the remaining scaffold portion of the DNA following incubation and cleavage by Eco81I, producing a shallower and/or faster event profile when passed through a nanopore. Panel C depicts an idealized current profile consistent with the remaining fragment that passes through a nanopore and that is not bound to the scaffold.

FIG. 12A depicts an example construct wherein a single fusion comprises two different enzyme cleavable linkers for detecting enzyme activity: a cleavable linker susceptible to proteolytic degradation by MMP9; and a specific sequence recognized and cleaved by the endonuclease Eco81I. FIG. 12B depicts the process of cleavage of the DNA sequence linker by the presence of active endonuclease Eco81I, while the cleavable linker remains intact in the absence of active MMP9. Upon incubation of the full scaffold/fusion/payload construct with a sample containing Eco81I but absent MMP9, the specific sequence recognized by the endonuclease is cleaved, resulting in two separate fragments. FIG. 12C depicts idealized nanopore event signatures comparing (i) the full molecular construct, with (ii, iii) the fragments following Eco81I cleavage of the cleavable linker.

FIG. 13 demonstrates the conjugation of a protease sensitive molecular construct via an electrophoretic mobility shift assay (EMSA). A 500 bp double-stranded DNA scaffold (Lane 1) is tethered to a fusion comprising an MMP9 sensitive cleavable linker, which is also tethered to a payload molecule (Lane 2, upper band). For additional payload bulk, the protein monostreptavidin is bound to the payload portion of the complex (Lane 3, upper band).

FIG. 14 shows a gel comparing the electrophoretic mobility of the protease-sensitive construct before and after incubation with the protease MMP9. A 500 bp DNA scaffold is conjugated to a fusion containing the MMP9 cleavable linker, which is tethered to a payload (Lanes 1 and 3). Following incubation with MMP9, the construct shows an increase in electrophoretic mobility indicated by a shift down in DNA banding, indicative of full digestion of the cleavable linker (Lane 2).

FIG. 15 shows the resulting fragments of an endonuclease sensitive construct before and after degradation by the Saul isoschizomer Eco81I. Degradation by Eco81I of a site within a 500 bp DNA scaffold covalently linked to payload results in the complete hydrolysis at the encoded sequence CC/T(N)AGG (comprised within the fusion portion of the 500 bp DNA). Hydrolysis results in two fragments, 306 bp scaffold, and the fusion:payload comprising 194 bp DNA tethered to the payload (Lane 3).

FIG. 16 demonstrates the cleavage of MMP9 sensitive construct in a titration of human urine. A 300 bp scaffold tethered to a fusion comparison an MMP9 sensitive construct with payload bound (Lane 5) was incubated with MMP9 in the presence of increasing concentrations of urine ranging from 0 to 30%. Efficient cleavage occurs in up to 5% urine (Lane 2), while complete inhibition of the enzyme is apparent at >15% urine in solution (Lanes 3 and 4) as indicated by no change in the upper band that indicates intact DNA scaffold:fusion:payload.

FIG. 17 demonstrates single-molecule sensing with a nanopore device. (a) A representative current-shift event caused by a 3.2 kb dsDNA passing through a 27 nm diameter nanopore at voltage V=100 mV (1M LiCl). Events are quantitated by conductance shift depth (6G=61/V) and duration. (b) Scatter plot of 6G versus duration for 744 events recorded over 10 minutes.

FIG. 18 compares nanopore event characteristics for DNA alone (500 bp), DNA-payload, and DNA-payload-monostreptavidin (DNA-payload-MS). DNA-payload produces an increase in the number of events with δG>1 nS compared to DNA alone. The addition of MS to the DNA-payload is to further increase the depth and duration of event signatures, as observed in the (a) scatter plot of δG versus duration and (b) percentage of events with δG>1 nS.

FIG. 19 compares nanopore event for DNA scaffold alone (300 bp), DNA:fusion:payload, and DNA:fusion:payload post activity of MMP9 protease, with MMP9 cleavable linker included in the fusion molecule. The percentage of events longer than 0.1 ms provides the signature with which to detect activity of the MMP9 enzyme with 99% confidence.

FIG. 20 compares nanopore event for DNA alone (500 bp), scaffold:fusion:payload, and scaffold:fusion:payload following incubation with Eco81I endonuclease, with a DNA cleavable linker for Eco81I included in the fusion portion of the DNA. The percentage of events longer than 0.06 ms provides the signature with which to detect activity of the Eco81I enzyme with 99% confidence.

### DETAILED DESCRIPTION

Throughout this application, the text refers to various embodiments of the present devices, compositions, systems, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present invention.

Also throughout this disclosure, various publications, patents and published patent specifications are referenced by an identifying citation. The disclosures of these publications, patents and published patent specifications are hereby incorporated by reference into the present disclosure in their entireties

As used herein, the term “comprising” is intended to mean that the systems, devices, and methods include the recited components or steps, but not excluding others. “Consisting essentially of when used to define systems, devices, and methods, shall mean excluding other components or steps of any essential significance to the combination. “Consisting of shall mean excluding other components or steps. Embodiments defined by each of these transition terms are within the scope of this invention.

All numerical designations, e.g., distance, size, temperature, time, voltage and concentration, including ranges, are approximations which are varied (+) or (−) by increments of 0.1. It is to be understood, although not always explicitly stated that all numerical designations are preceded by the term “about”. It also is to be understood, although not always explicitly stated, that the components described herein are merely exemplary, and that equivalents of such are known in the art.

As used herein, “a device comprising a nanopore that separates an interior space” shall refer to a device having a pore that comprises an opening within a structure, the structure separating an interior space into two volumes or chambers. The device can also have more than one nanopore, and with one common chamber between every pair of pores.

As used herein, the term “fusion molecule” refers to molecules or compounds that comprise a cleavable linker sensitive to enzymatic, photolytic, or chemical cleavage by a target molecule or target condition suspected to be present in a sample. The fusion also binds to a polymer scaffold and a payload molecule. Upon translocation through the nanopore, the current signature determines if the payload molecule is bound to the polymer scaffold or not. In this way, cleavage of the cleavable linker within the fusion molecule may be detected and/or quantified.

As used herein the term “cleavage” or refers to a process or condition that breaks a chemical bond to separate a molecule or compound into simpler structures. A molecule (e.g., an enzyme) or a set of conditions, (e.g., photolysis), when in contact with a cleavable linker as described herein, can result in cleavage of the linker to generate a cleaved linker. As used herein specific cleavage refers to a known relationship between the linker and a target enzyme or condition, wherein the target molecule or condition is known to cleave the cleavable linker. Thus, the molecule or target specifically cleaves the linker when the cleavage of the linker can be used to infer the presence of the target molecule or condition.

As used herein, the term “cleavable linker” or “labile linker” refers to a substrate linker sensitive to enzymatic, photolytic, or chemical cleavage by a target molecule or condition. In some embodiments, the cleavable linker can be a deoxyribonucleic acid (DNA), a polypeptide, a carbon-oxygen bond, a carbon-sulfur bond, a carbon-nitrogen bond, or a carbon-carbon bond. In some embodiments, the cleavable linker sensitive to photolytic cleavage can be an ortho-nitrobenzyl derivative or phenacyl ester derivative. In some embodiments, the cleavable linker sensitive to chemical cleavage can be an azo compounds, disulfide bridge, sulfone, ethylene glycolyl disuccinate, hydrazone, acetal, imine, vinyl ether, vicinal diol, or picolinate ester.

In some embodiments, the term “cleavable domain” as used herein refers to a domain of a molecule that is sensitive to enzymatic, photolytic, or chemical cleavage by a target molecule or condition. Cleavable domain may be used interchangeably with cleavable linker when the cleavable domain is a component of the same type of molecule as the polymer scaffold or payload molecule. For example, in embodiments wherein the cleavable domain is on the polymer scaffold, one may also conceive of the polymer scaffold as comprising a polymer scaffold and a fusion molecule comprising a cleavable linker (i.e., the cleavable domain), wherein the fusion molecule is bound to the polymer scaffold, even though both the fusion molecule and the polymer scaffold are the same type of molecule (e.g., dsDNA).

As used herein, the term “target molecule” is the molecule of interest to be detected in a sample, and refers to a molecule (e.g., a hydrolase or lyase) capable of cleaving (e.g., through enzymatic cleavage) a cleavable linker region or domain. The target molecule may be detected by a method described herein through its cleavage of the cleavable linker within the fusion molecule bound to a polymer scaffold that translocates through a nanopore, providing a defined current impedance or current signature.

As used herein, the term “target condition” refers to a condition capable of photolytically modifying the cleavable linker via exposure to light within the wavelength range of 10 nm to 550 nm. Alternatively, the target condition may be capable of chemically modifying the cleavable linker via exposure to nucleophilic or basic reagents, electrophilic or acidic reagents, reducing reagents, oxidizing reagents, or an organometallic compound.

As used herein, the term “scaffold” or “polymer scaffold” refers to a negatively or positively charged polymer that translocates through a nanopore upon application of voltage. In some embodiments, a polymer scaffold comprises a cleavable domain or cleavable linker. In some embodiments, a polymer scaffold capable of binding or bound to a fusion molecule comprising a cleavable linker and translocating through a pore upon application of voltage. In some aspects, the polymer scaffold comprises a deoxyribonucleic acid (DNA), a ribonucleic acid (RNA), a peptide nucleic acid (PNA), a DNA/RNA hybrid, or a polypeptide. The scaffold may also be a chemically synthesized polymer, and not a naturally occurring or biological molecule. In a preferred embodiment, the polymer scaffold is dsDNA to allow more predictable signals upon translocation through the nanopore and reduce secondary structure present in ssDNA or RNA. In some embodiments, the polymer scaffold comprises a fusion molecule binding site that may reside on the end of the scaffold, or at both ends of the scaffold. The scaffold and fusion molecule may be connected via a covalent bond, a hydrogen bond, an ionic bond, a van der Waals force, a hydrophobic interaction, a cation-pi interaction, a planar stacking interaction, or a metallic bond. Alternatively, direct covalent tethering of the cleavable linker component to the scaffold may connect the scaffold and the fusion molecule. Alternatively, a connector component of the fusion may join the cleavable linker to the scaffold via direct covalent tethering. In a preferred embodiment, the fusion molecule comprises a scaffold-binding domain can be a DNA, RNA, PNA, polypeptide, a cholesterol/DNA hybrid, or a DNA/RNA hybrid.

As used herein, the term “payload” refers to molecules or compounds that are bound to the fusion molecule to enhance selectivity and/or sensitivity of detection in a nanopore. In some embodiments, the payload molecule can be a dendrimer, double stranded DNA, single stranded DNA, a DNA aptamer, a fluorophore, a protein, a polypeptide, a nanorod, a nanotube, fullerene, a PEG molecule, a liposome, or a cholesterol-DNA hybrid. In preferred embodiments, the cleavable linker and the payload are connected directly or indirectly via a covalent bond, a hydrogen bond, an ionic bond, a van der Waals force, a hydrophobic interaction, a cation-pi interaction, a planar stacking interaction, or a metallic bond. The payload adds size to the scaffold:fusion molecule, and facilitates detection of cleavage of the cleavable linker, with scaffold:fusion:payload having a markedly different current signature when passing through the nanopore, than the remaining scaffold:fusion and fusion:payload components following cleavage of the cleavable linker (e.g., cleavage of the cleavable linker by a hydrolyzing enzyme).

As used herein, the term “binding domain” refers to a domain of a molecule that specifically binds to another molecule in the presence of that molecule. In some embodiments, disclosed herein are a polymer scaffold binding domain that binds specifically to a polymer scaffold, and a payload molecule binding domain that binds specifically to a payload molecule.

As used herein, the term “connector” refers to a molecule that acts to bridge two molecules spatially apart from one another, allowing them to be bound through the connector. In some embodiments, polyethylene glycol (PEG) can act as a connector between, e.g., a fusion molecule and a polymer scaffold or payload molecule.

As used herein, the term “nanopore” refers to an opening (hole or channel) of sufficient size to allow the passage of particularly sized polymers. With an amplifier, voltage is applied to drive negatively charged polymers through the nanopore, and the current through the pore detects if molecules are passing through it.

As used herein, the term “sensor” refers to a device that collects a signal from a nanopore device. In many embodiments, the sensor includes a pair of electrodes placed at two sides of a pore to measure an ionic current across the pore when a molecule or other entity, in particular a polymer scaffold, moves through the pore. In addition to the electrodes, an additional sensor, e.g., an optical sensor, may be to detect an optical signal in the nanopore device. Other sensors may be used to detect such properties as current blockade, electron tunneling current, charge-induced field effect, nanopore transit time, optical signal, light scattering, and plasmon resonance.

As used herein, the term “current measurement” refers to a series of measurements of current flow at an applied voltage through the nanopore over time. The current is expressed as a measurement to quantitate events, and the current normalized by voltage (conductance) is also used to quantitate events.

As used herein, the term “open channel” refers to the baseline level of current through a nanopore channel within a noise range where the current does not deviate from a threshold of value defined by the analysis software.

As used herein, the term “event” refers to a set of current impedance measurements that begins when the current measurement deviates from the open channel value by a defined threshold, and ends when the current returns to within a threshold of the open channel value.

As used herein, the term “current impedance signature” refers to a collection of current measurements and/or patterns identified within a detected event. Multiple signatures may also exist within an event to enhance discrimination between molecule types.

Detecting Enzyme Activity

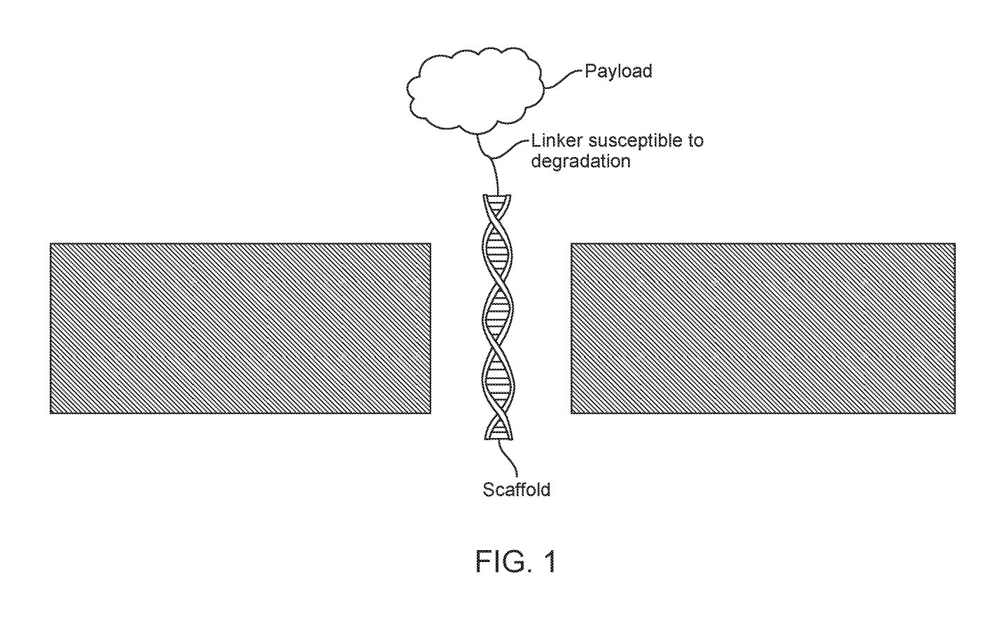

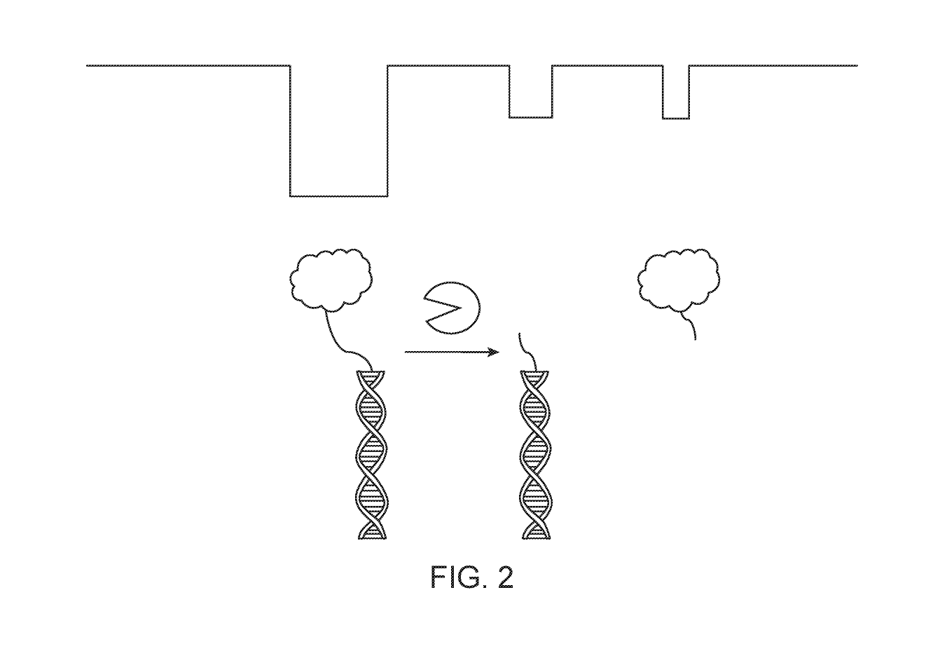

Provided herein are methods and compositions for detecting enzymatic activity using a modified cleavable linker. As shown in FIG. 1, a molecule designed for detecting the presence of enzymatic activity a scaffold, a payload, and a fusion comprising a linker susceptible to degradation. This scaffold:fusion(linker):payload molecule can be used in a nanopore system to detect the presence of enzymatic activity in a sample. In particular, FIG. 2 provides a conceptual example showing the method of using the molecule (FIG. 1) with a nanopore to detect the presence of enzymatic activity. In FIG. 2, the cleavable linker is a polypeptide sequence that is the substrate of a protease. If protease is absent from the sample, the scaffold/fusion(linker)/payload molecule will remain intact and generate a longer and deeper signal upon translocation through the nanopore under an applied voltage. However, if the protease of interest is present in the sample and is active, it will digest the cleavable linker polypeptide sequence, generating a separate payload and scaffold molecule, each of which will generate a unique current blockade signature when these molecules pass through the nanopore under an applied voltage. Current blockades and resolution can be adjusted by varying the applied voltage, and other conditions (salt concentration, pH, temperature, nanopore geometry, nanopore material, etc.). Resolution of enzymatic activity can also be adjusted by adjusting the concentration of the target molecule in solution in contact with the nanopore.

Cleavable linkers can come in a variety of forms. It is the specificity of a target enzyme for its substrate that gives our nanopore activity assays its specificity. That is, background molecules from the sample are unlikely to appreciably modify or cut the cleavable linker, while the target molecule or condition has a high affinity for cutting and/or modifying the substrate to the extent that nanopore measurements can resolve and detect the cutting and/or modification.

A payload molecule, as described herein, can be any molecule that aids in detection of modification (e.g., cleavage) of the cleavable linker molecule in the nanopore. This can include, for example, a dendrimer, a DNA aptamer, a fluorophore, a protein, or a polyethylene glycol (PEG) polymer.

The cleavable linker within the fusion component of the scaffold:fusion:payload construct can include any substrate that is the substrate of the activity of the target enzyme of interest. This can include, for example, a polypeptide sequence, a nucleotide sequence, or any other enzymatic substrate. This linker may also be susceptible to cleavage by environmental conditions (e.g., pH, UV, and/or light).

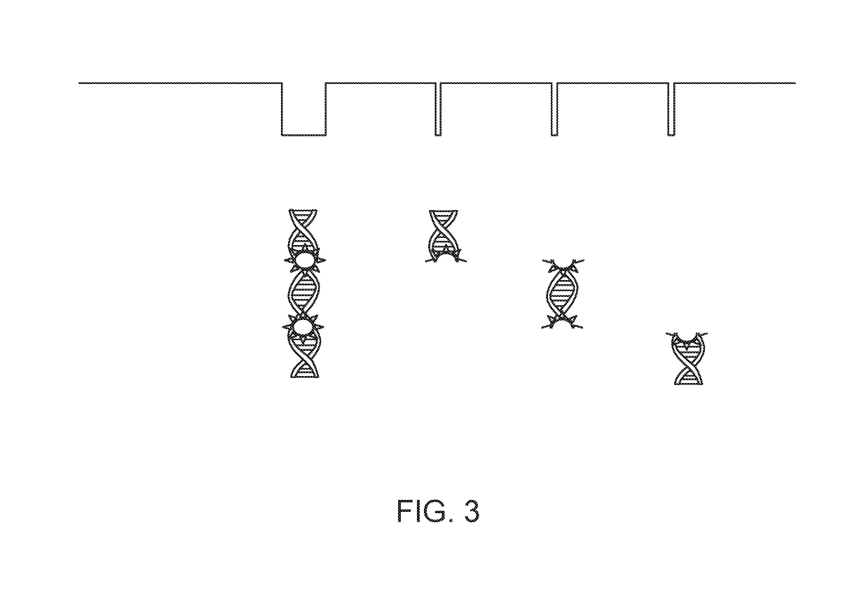

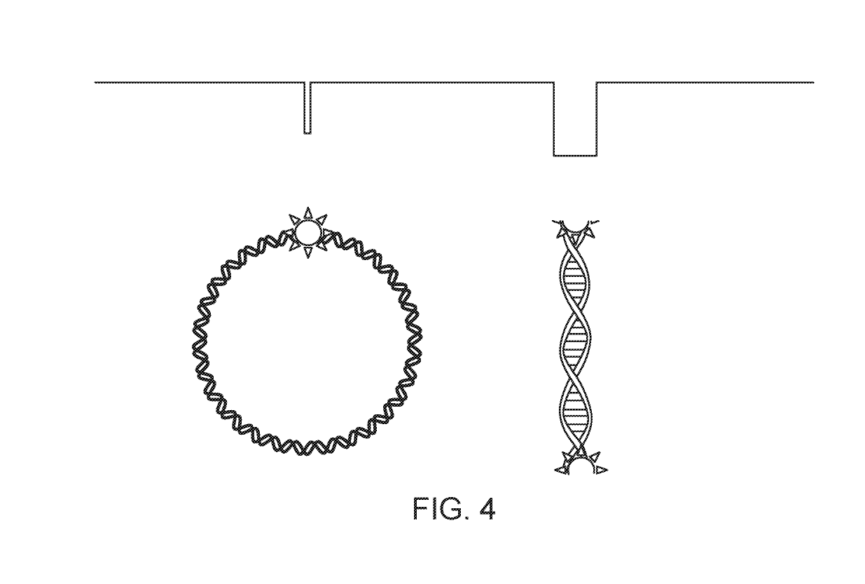

In another embodiment, the scaffold:fusion:payload could be reduced to only a scaffold construct, particularly, when the cleavable linker comprises a polynucleotide sequence. In such embodiments, the scaffold is comprises double-stranded DNA. This is relevant, for example, to detect bacterial contamination by detection of endonuclease activity as shown in FIGS. 3 and 4. An endonuclease target comprising a DNA sequence within a DNA scaffold is provided in solution in contact with the nanopore. In the absence of the endonuclease, a longer current signature occurs during the translocation of each target molecule. Upon addition of a sample containing the endonuclease of interest, the target molecule is digested, resulting in shorter current signatures as the digested DNA fragments translocate through the nanopore with an applied voltage, and a decrease in the current signature duration from the full length target sequence. Linear (FIG. 3) or circular (FIG. 4) scaffold molecules may be used for detection of endonuclease activity.

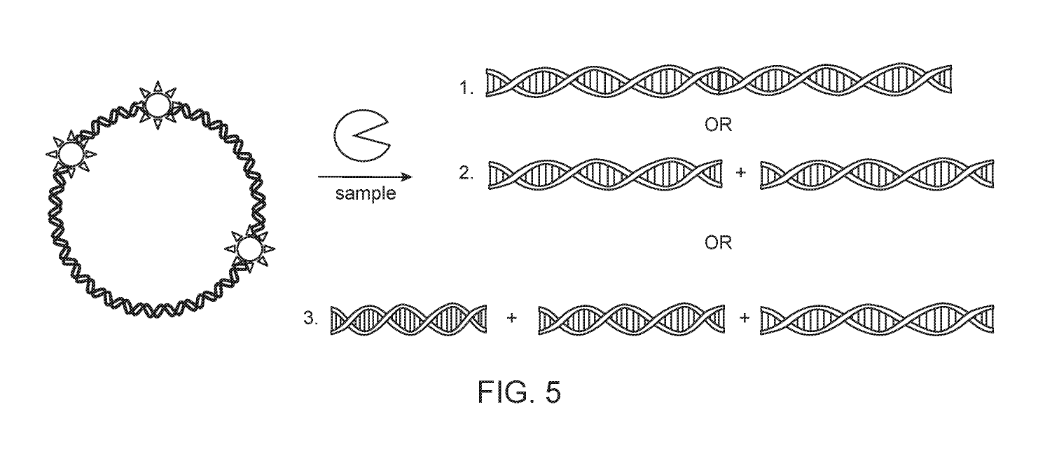

In another embodiment, the scaffold construct can be used to perform multiplexed detection of bacterial contamination by endonuclease activity. One example of such a method is shown in FIG. 5. In this example, the cleavable domain-containing scaffold comprises multiple unique cleavable domains for digestion by one or more target endonucleases of interest. The resulting fragments are then detected in solution by the nanopore system. Translocation of the digested fragments under applied voltage provides unique current signatures through an appropriately sized pore, allowing detection of which sites had been digested, and therefore, which endonucleases are present in the sample of interest.

In other embodiments, the scaffold:fusion:payload construct as shown in FIGS. 1-2 may also be used to detect endonuclease activity. In that case, a fusion molecule comprises the target DNA sequence, and attaches to a payload that facilitates nanopore detection of digestion.

Multiplexing can be achieved in varying ways, e.g., by attaching more than one fusion:payload to each scaffold molecule. With this construct, a single pore device may be able to detect the activity of multiple target molecules (e.g., enzymes) or target conditions, for an appropriately designed scaffold and fusion:payload(s). Alternatively, loading the scaffold into a two-pore device (PCT Publication No. WO/2013/012881, incorporated by reference herein in its entirety) could be used to assay the activity of multiple target molecules (e.g., enzymes) or target conditions.

The “activity status” of a target molecule or target condition, as used herein, refers to whether the cleavable linker within the fusion molecule is intact (resulting in a full scaffold:fusion:payload complex) or not (resulting in scaffold molecules not bound to payload molecules). Essentially, the activity status can be one of these two potential statuses.

Detection of the activity status of a target molecule or target condition can be carried out by various methods. In one aspect, by virtue of the different sizes of molecules at each status, when the scaffold:fusion:payload complex passes through the pore, the current signature will be sufficiently distinct from when scaffold alone or payload alone pass through the pore. In one aspect, with a positive voltage applied and KCI concentrations greater than 0.4 M or LiCl concentrations greater than 0.2M in the experiment buffer, the measured current signals (FIG. 2) are downward and thus attenuations. The three signals in FIG. 2 can be differentiated from one another by the amount of the current shift (depth) and/or the duration of the current shift (width), or by any other feature in the signal that differentiates the three event types.

In another aspect, with a positive voltage applied and KCI concentrations less than 0.4M in the experiment buffer, the measured current signals may have current enhancements for scaffold or any component of the complex comprised of DNA. This was shown for DNA alone in the published research by Smeets, Ralph M M, et al. “Salt dependence of ion transport and DNA translocation through solid-state nanopores.” Nano Letters 6.1 (2006): 89-95. In this case, the three signal types can be differentiated by the event amplitude direction (polarity) relative to the open channel baseline current level (408), in addition to the three signals commonly having different amounts of the current shift (height) and/or the duration of the current shift (width), or by any other feature in the signal that differentiates the three event types.

In aspects of the FIG. 2 embodiments, the sensor comprises electrodes, which are connected to power sources and can detect the current. Either one or both of the electrodes, therefore, serve as a “sensor.” In this embodiment, a voltage-clamp or a patch-clamp is used to simultaneously supply a voltage across the pore and measure the current through the pore.

In some aspects, a payload is added to the complex to aid detection. In one aspect, the payload includes a charge, either negative or positive, to facilitate detection. In another aspect, the payload adds size to facilitate detection. In another aspect, the payload includes a detectable label, such as a fluorophore, which can be detected with an optical sensor focused at the site of nanopore translocation, for example.

Polymer Scaffold

A polymer scaffold suitable for use in the present technology is a scaffold that can be loaded into a nanopore device and passed through the pore from one end to the other.

Non-limiting examples of polymer scaffolds include nucleic acids, such as deoxyribonucleic acid (DNA), ribonucleic acid (RNA), or peptide nucleic acid (PNA), dendrimers, and linearized proteins or peptides. In some aspects, the DNA or RNA can be single-stranded or double-stranded, or can be a DNA/RNA hybrid molecule.

In a preferred embodiment, double stranded DNA is used as a polymer scaffold. There are several advantages of dsDNA over ssDNA as a polymer scaffold. In general, non-specific interactions and unpredictable secondary structure formation are more prevalent in ssDNA, making dsDNA more suitable for generating reproducible current signatures in a nanopore device. Also, ssDNA elastic response is more complex than dsDNA, and the properties of ssDNA are less well known than for dsDNA. Therefore, many embodiments of the invention are engineered to encompass dsDNA as a polymer scaffold, including one or more of the payload and/or fusion molecules used herein.

In one aspect, the polymer scaffold is synthetic or chemically modified. Chemical modification can help to stabilize the polymer scaffold, add charges to the polymer scaffold to increase mobility, maintain linearity, or add or modify the binding specificity, or add chemically reactive sites to which a fusion and/or payload can be tethered. In some aspects, the chemical modification is acetylation, methylation, summolation, oxidation, phosphorylation, glycosylation, thiolation, addition of azides, or alkynes or activated alkynes (DBCO-alkyne), or the addition of biotin.

In some aspects, the polymer scaffold is electrically charged. DNA, RNA, PNA and proteins are typically charged under physiological conditions. Such polymer scaffolds can be further modified to increase or decrease the carried charge. Other polymer scaffolds can be modified to introduce charges. Charges on the polymer scaffold can be useful for driving the polymer scaffold to pass through the pore of a nanopore device. For instance, a charged polymer scaffold can move across the pore by virtue of an application of voltage across the pore.

In some aspects, when charges are introduced to the polymer scaffold, the charges can be added at the ends of the polymer scaffold. In some aspects, the charges are spread uniformly over the polymer scaffold.

Scaffold:Fusion:Payload Construction

In a preferred embodiment, the fusion molecule contains: 1) the cleavable linker, 2) the scaffold attachment site, and 3) a payload attachment site.

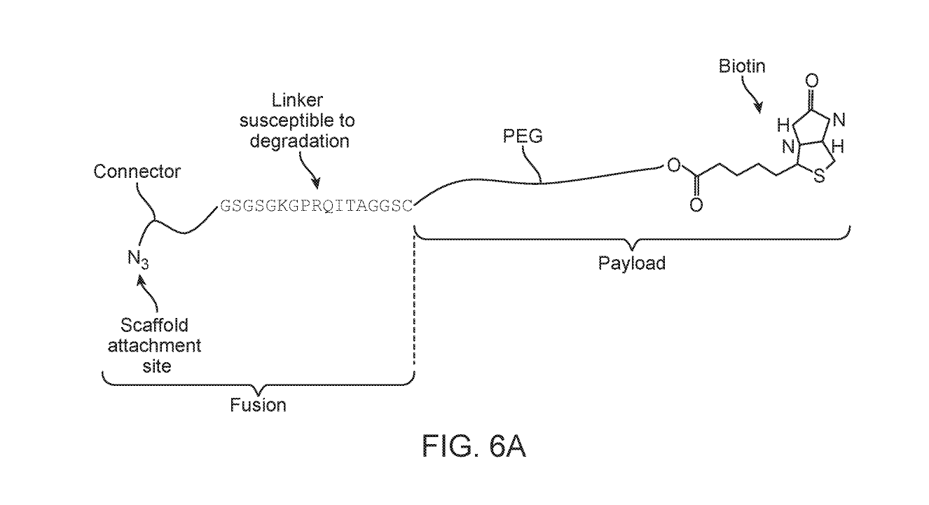

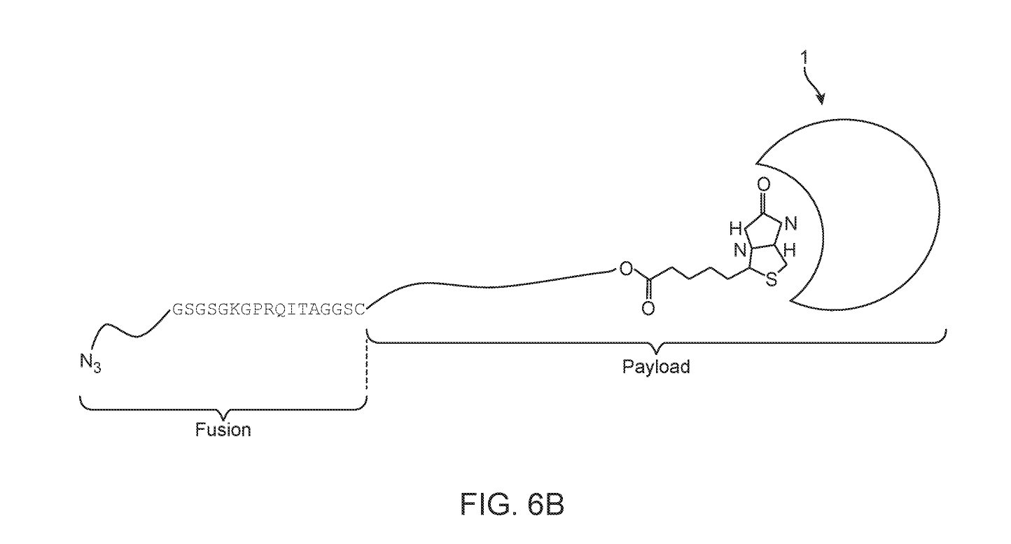

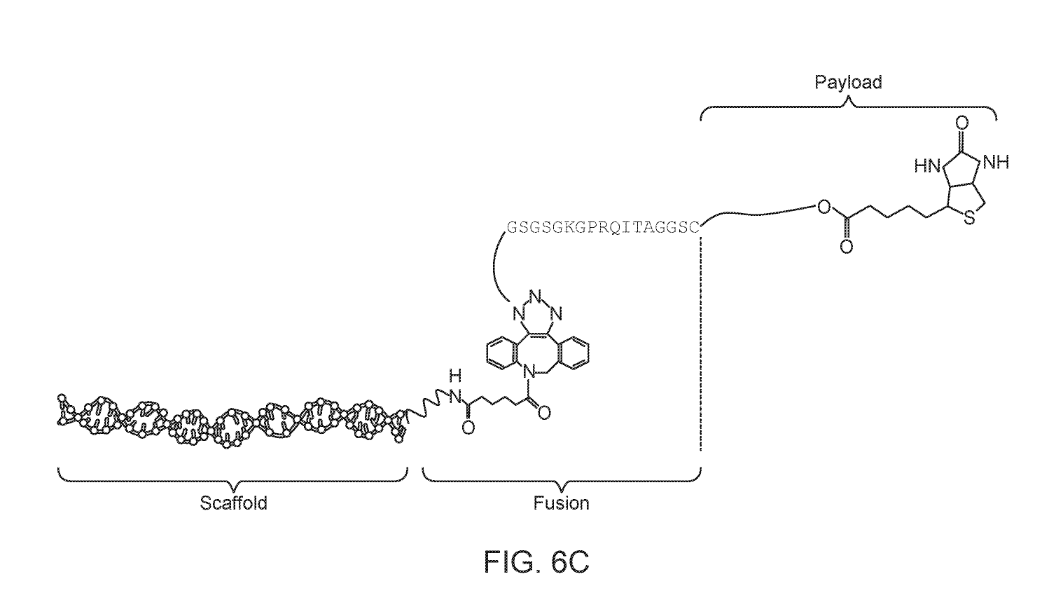

In a preferred embodiment, a representative example of a fusion:payload is shown in FIG. 6. Specifically, FIG. 6A shows a fusion with the following components, from left-to-right: an azide chemical handle for attachment to the scaffold; the connector PEG₄; a flexible Gly-Ser motif; MMP9-sensitive peptide sequence SGKGPRQITA; and a flexible Gly-Ser motif for attachment to the payload. FIG. 6A shows a payload with the following components, from left-to-right: Cys-5 kDa PEG, and a biotin. The option of adding bulk to the payload to facilitate activity detection is made possible by binding monostreptavidin to the biotin site (FIG. 6B). The added bulk can produce a more distinct signature difference between scaffold:fusion:payload, prior to enzyme activity, and scaffold alone and payload alone following enzyme activity.

In this embodiment, the cleavable linker peptide sequence in this example is SGKGPRQITA. This peptide had previously been identified as highly sensitive to MMP9 activity (Kridel, Steven J., et al. “Substrate hydrolysis by matrix metalloproteinase-9. Journal of Biological Chemistry 276.23 (2001): 20572-20578).

In this embodiment, attachment to a DNA scaffold can be achieved in a variety of ways. In this example (FIG. 6), the DNA could be generated using a dibenzocyclooctyne (DBCO) modified primer, effectively labeling all DNA scaffold molecules with a DBCO chemical group to be used for conjugation purposes via copper-free “click” chemistry to the azide-tagged fusion molecule, producing the full scaffold:fusion:payload complex (FIG. 6C).

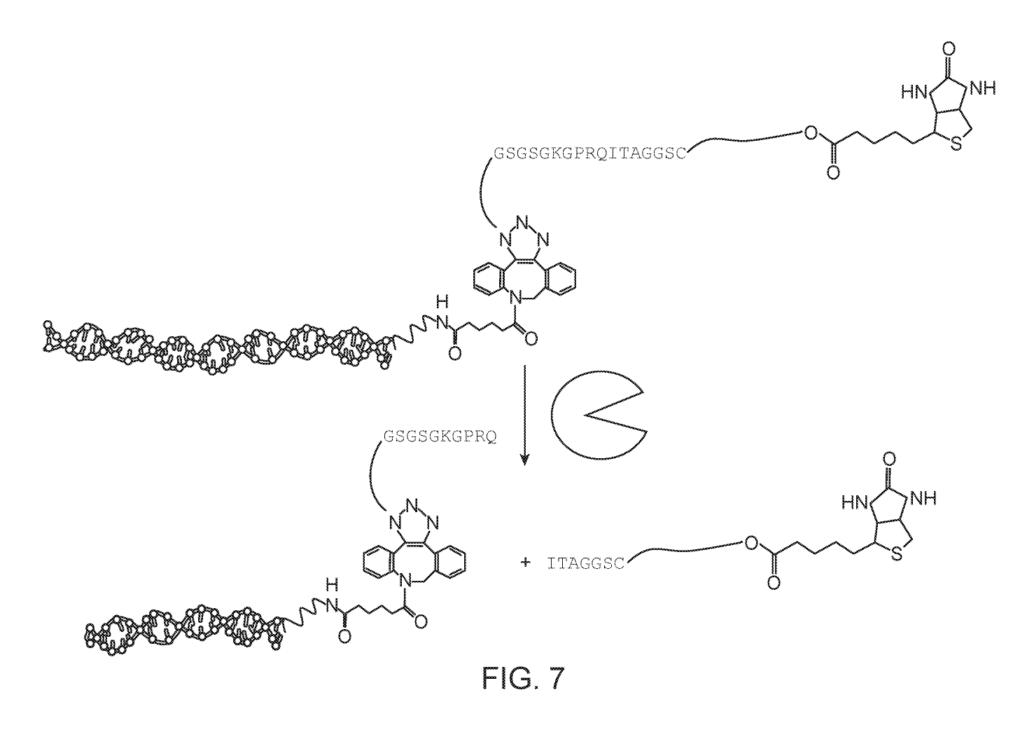

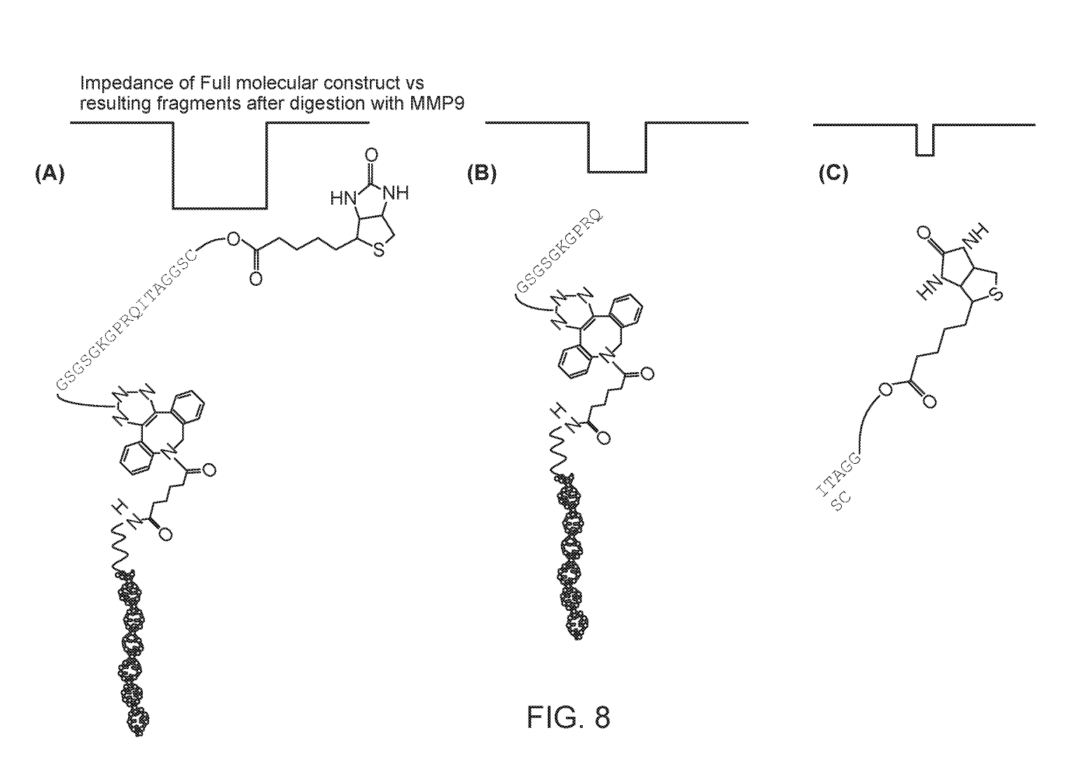

For the representative example (FIG. 6), MMP9 activity can be assayed by combining a sample containing MMP9 with the scaffold:fusion:payload reagent, and after a period sufficient for activity to come to completion, and in conditions that permit activity (FIG. 7), the combined reagents can be measured with the nanopore (FIG. 8). Activity is assayed by single molecule measurements afforded by the nanopore, with full complex producing the deeper and longer event signature, while products producing faster and/or shallower event signatures, as depicted in FIG. 8.

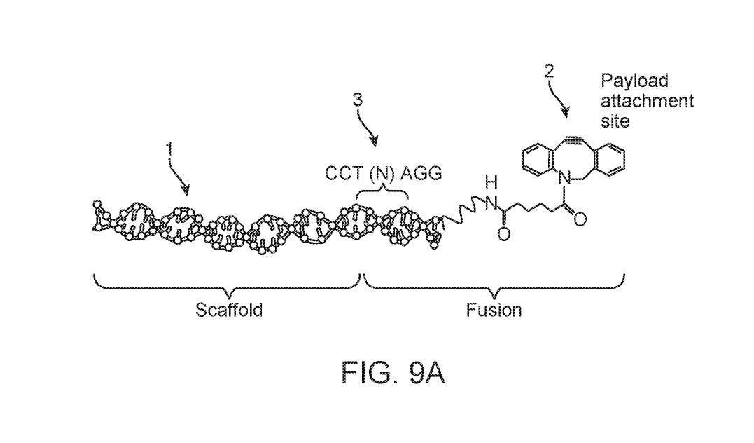

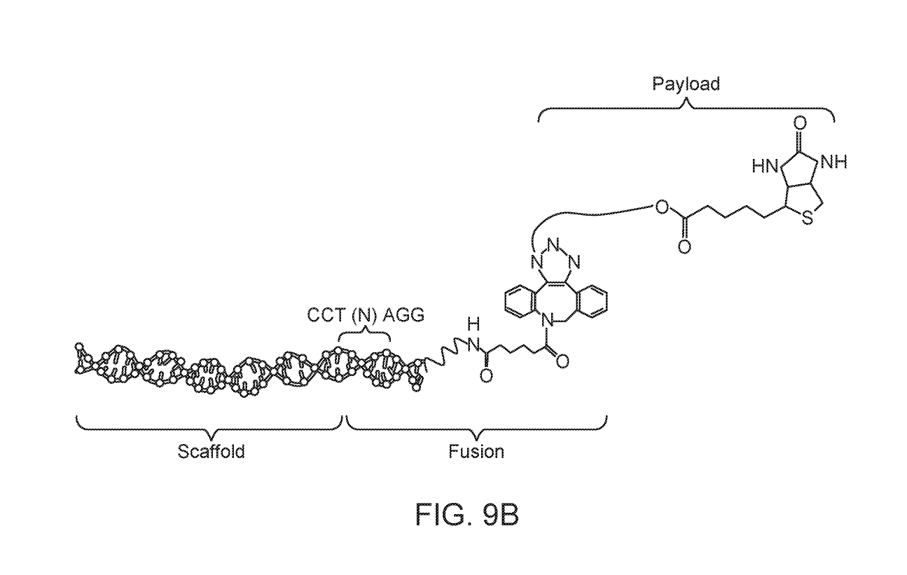

In another preferred embodiment, a representative example of a scaffold:fusion:payload is shown in FIG. 9. Specifically, FIG. 9A shows a scaffold:fusion with the following components, from left-to-right: (1) a scaffold comprising DNA; a fusion comprising (3) a DNA sequence that is susceptible to hydrolytic degradation by an endonuclease, and with (2) a dibenzocyclooctyne (DBCO) handle that can be used to conjugate to an azide bearing molecule as a payload (not shown). The DNA sequence that is susceptible to hydrolytic degradation is a Saul recognition sequence. FIG. 9B shows a payload bound to the molecule from FIG. 9A, comprising a Cys-5 kDa PEG and a biotin.

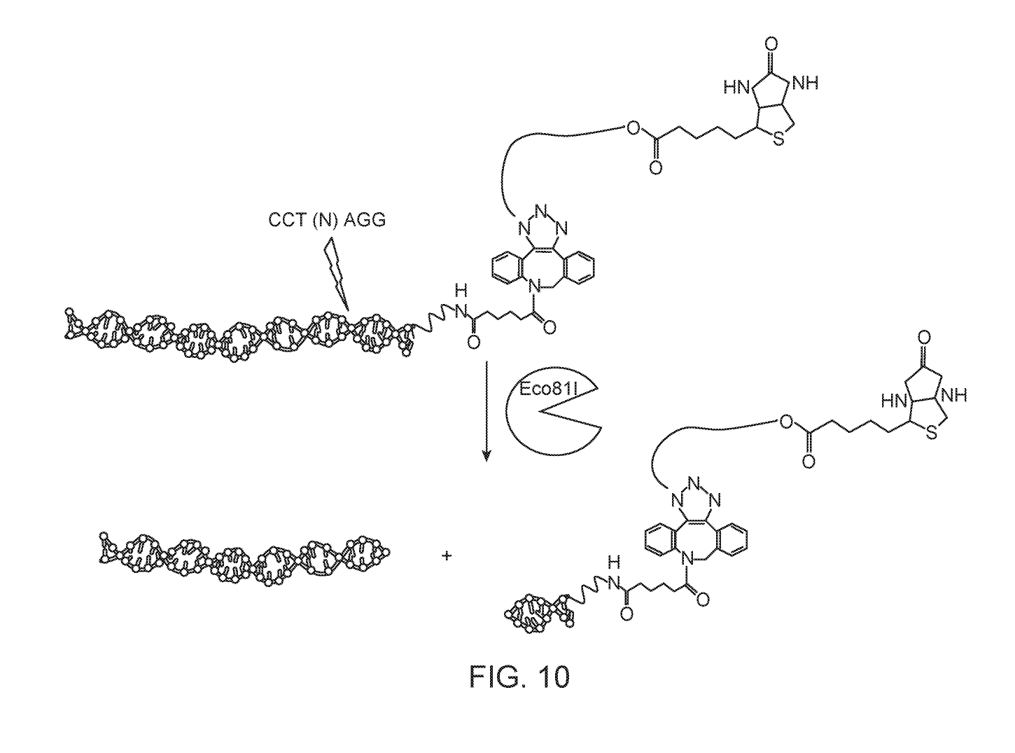

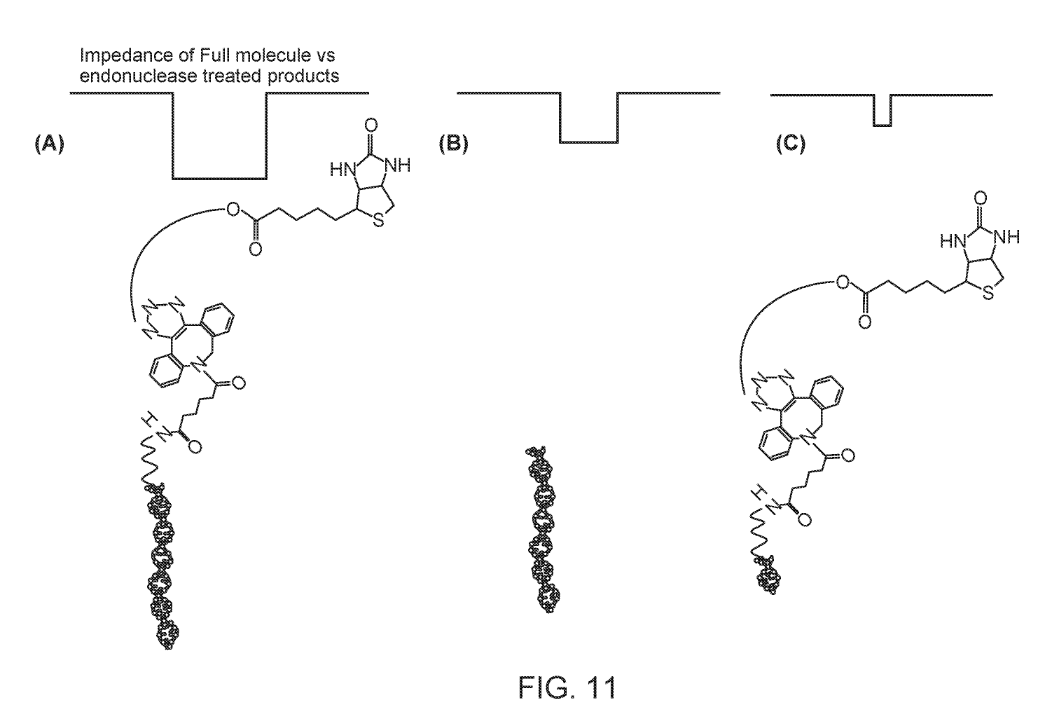

For the representative example (FIG. 9), MMP9 activity can be assayed by combining a sample containing Eco81I with the scaffold:fusion:payload reagent, and after a period sufficient for activity to come to completion, and in conditions that permit activity (FIG. 10), the combined reagents can be measured with the nanopore (FIG. 11). Upon exposure to the Saul isoschizomer Eco81I, the molecular construct is hydrolyzed at the DNA sequence CCT(N)AGG, thereby cleaving the construct in two. Activity is assayed by single molecule measurements afforded by the nanopore, with full complex producing the deeper and longer event signature, while products producing faster and/or shallower event signatures, as depicted in FIG. 11.

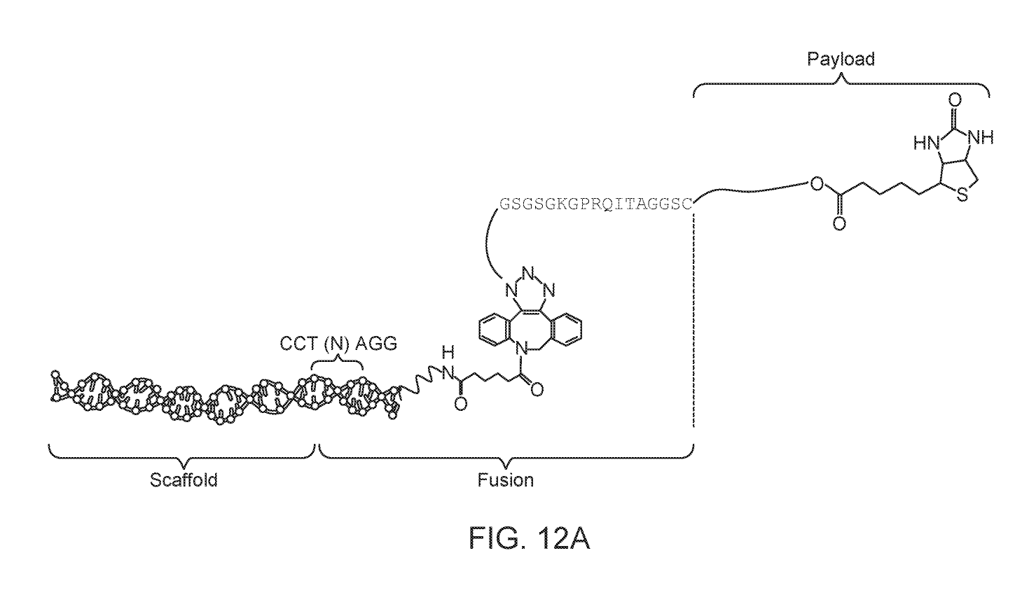

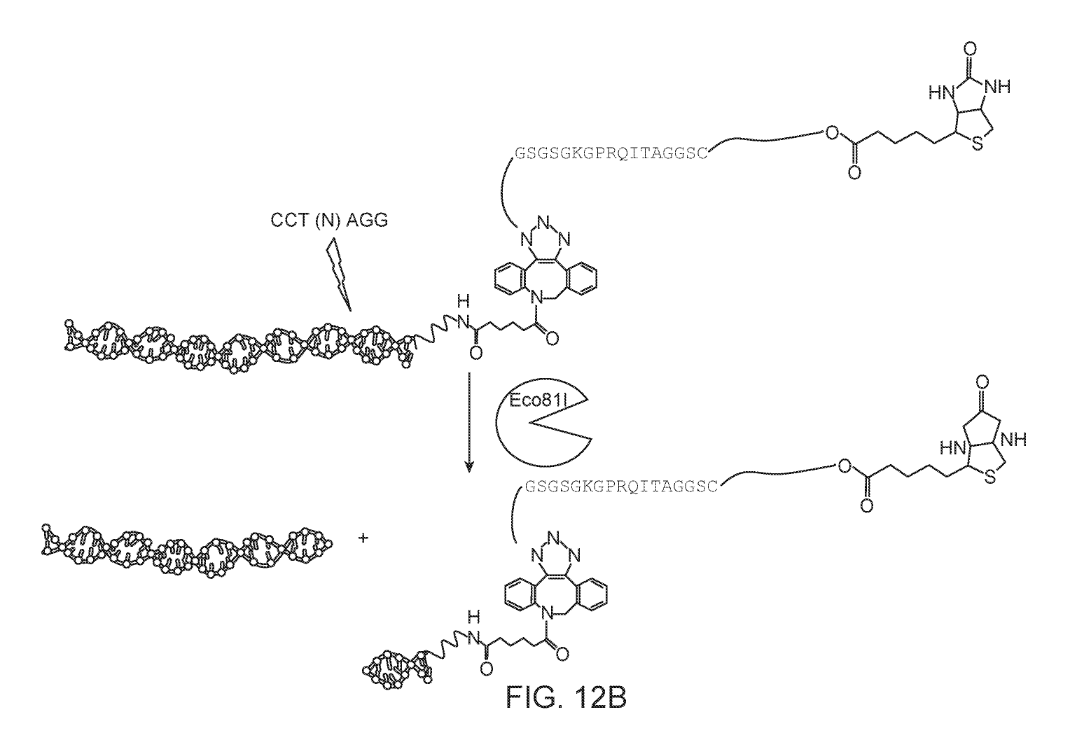

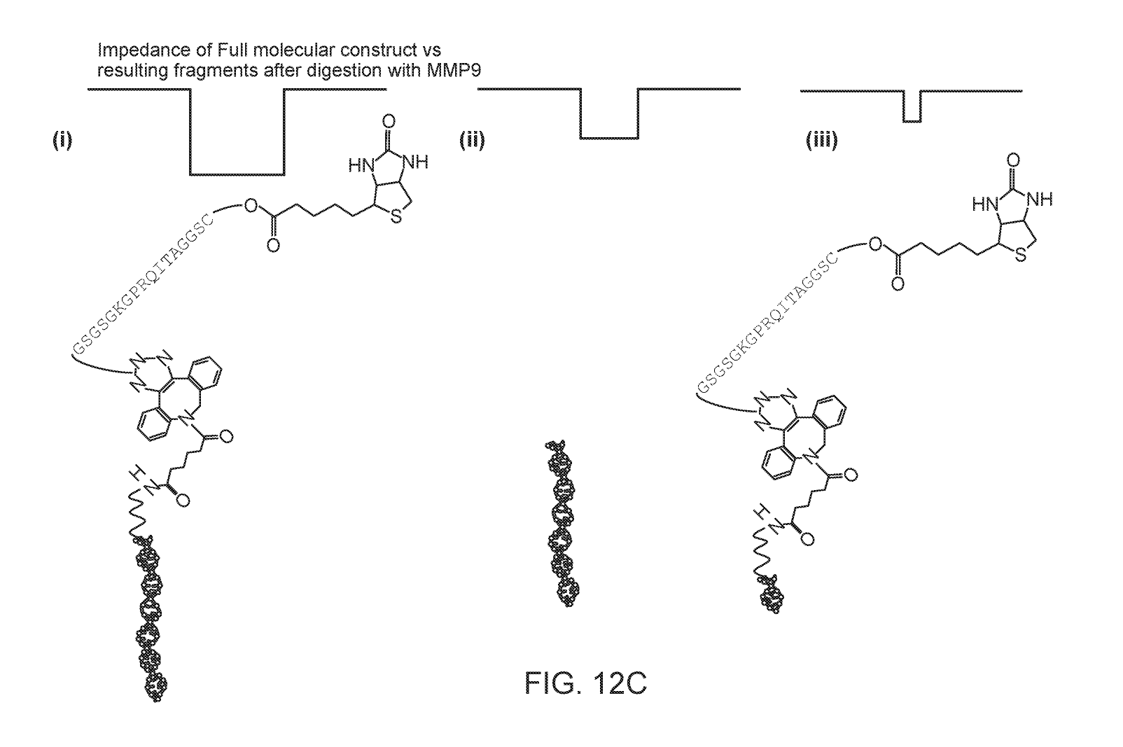

In another embodiment, the fusion molecule of the scaffold:fusion:payload construct comprises two or more cleavable linkers for detecting and quantitating enzyme activity. In a representative example (FIG. 12), the fusion comprises: i) the DNA sequence CCT(N)AGG that is susceptible to hydrolytic degradation by the endonuclease Eco81I, and that is adjacent to the DNA scaffold, and ii) the MMP9-sensitive peptide sequence SGKGPRQITA. In this way, a single reagent could be used to detect the presence of either MMP-9 or Eco81I.

In another embodiment, the scaffold-attachment site of the fusion molecule can be a nucleic acid or a polypeptide that is itself a scaffold-binding domain. In some embodiments, the scaffold-binding domain of the fusion is a peptide sequence forming a functional portion of a protein, although the binding domain does not have to be a protein. For nucleic acids, for instance, there are proteins that specifically recognize and bind to sequences (motifs) such as promoters, enhancers, thymine-thymine dimers, and certain secondary structures such as bent nucleotide and sequences with single-strand breakage.

In some aspects, the scaffold-domain of the fusion includes a chemical modification that causes or facilitates recognition and binding. For example, methylated DNA sequences can be recognized by transcription factors, DNA methyltransferases or methylation repair enzymes. In other embodiments, biotin may be incorporated into, and recognized by, avidin family members. In such embodiments, biotin forms the fusion binding domain and avidin or an avidin family member is the polymer scaffold-binding domain on the fusion. Due to their binding complementarity, fusion binding domains and polymer scaffold domains may be reversed so that the fusion binding domain becomes the polymer scaffold binding domain, and vice versa.

Molecules, in particular proteins, that are capable of specifically recognizing nucleotide binding motifs are known in the art. For instance, protein domains such as helix-turn-helix, a zinc finger, a leucine zipper, a winged helix, a winged helix turn helix, a helix-loop-helix and an HMG-box, are known to be able to bind to nucleotide sequences.

In some aspects, the fusion binding domains can be locked nucleic acids (LNAs), bridged nucleic acids (BNA), Protein Nucleic Acids of all types (e.g. bisPNAs, gamma-PNAs), transcription activator-like effector nucleases (TALENs), clustered regularly interspaced short palindromic repeats (CRISPRs), or aptamers (e.g., DNA, RNA, protein, or combinations thereof).

In some aspects, the fusion binding domains are one or more of DNA binding proteins (e.g., zinc finger proteins), antibody fragments (Fab), chemically synthesized binders (e.g., PNA, LNA, TALENS, or CRISPR), or a chemical modification (i.e., reactive moieties) in the synthetic polymer scaffold (e.g., thiolate, biotin, amines, carboxylates).

In some embodiments, the polymer scaffold includes a sequence of fusion-binding domains which are used for multiplexing enzyme activity, with each domain having a unique fusion:payload comprising a unique cleavable linker for a target enzyme of interest.

Target Molecules and Conditions

Enzymatic activity present in a sample can indicate the presence of toxins, a disorder, or other condition of an organism. For example, proteases are critically important molecules found in humans that regulate a wide variety of normal human physiological processes including wound healing, cell signaling, and apoptosis. Because of their critical role within the human body, abnormal protease activity has been associated with a number of disease states including, but not limited to, rheumatoid arthritis, Alzheimer's disease, cardiovascular disease and a wide range of malignancies. Proteases are found in nearly all human fluids and tissue, and their activity levels can signal the presence of a condition.

The value of our assay is that it provides a single-molecule method of detecting the presence of any active enzyme (including proteases) that cleaves its associated specific cleavable linker by breaking a chemical bond, e.g., by hydrolysis or some other means.

The target molecule capable of enzymatically modifying its cleavable linker region can be a hydrolase. In some embodiments, the hydrolase can be from the subclass of proteases, endonucleases, glycosylases, esterases, nucleases, phosphodiesterases, lipase, phosphatases, or any other subclass of hydrolases.

In other embodiments, the target molecule capable of enzymatically modifying its cleavable linker region can be a lyase. In some embodiments, the lyases can be from any one of seven subclasses: lyases that cleave carbon-carbon bonds, such as decarboxylases (Enzyme Commission (EC) 4.1.1), aldehyde lyases (EC 4.1.2), oxo acid lyases(EC 4.1.3) and others (EC 4.1.99); lyases that cleave carbon-oxygen bonds, such as dehydratases (EC 4.2); lyases that cleave carbon-nitrogen bonds (EC 4.3); lyases that cleave carbon-sulfur bonds (EC 4.4); lyases that cleave carbon-halide bonds (EC 4.5); lyases that cleave phosphorus-oxygen bonds, such as adenylate cyclase and guanylate cyclase (EC 4.6); and other lyases, such as ferrochelatase (EC 4.99).

In other embodiments, the cleavable linker region of the fusion molecule, within the scaffold:fusion:payload construct, is exposed to a target condition to be detected. In one embodiment, the target condition is capable of photolytically modifying the cleavable linker via exposure to light within the wavelength range of 10 nm to 550 nm.

Light exposure conditions that promote breaking of bonds within a cleavable linker can reveal environmental conditions that can be correlated with a number of different health hazards, conditions, or disease-causing or disease-promoting states.

Ultraviolet (UV) light coming from the sun is known to strongly correlate with a variety of human conditions, and depletion of the ozone in the stratosphere over time is thought to lead to increased levels of ultraviolet radiation that reaches the surface of the Earth.

UV radiation is cumulative over the span of one's life, and has been shown to be a major contributing factor to melanoma, a deadly form of skin cancer. Additionally, UV light has profound effects on the human eye, and has been shown to increase retinal degradation as well as be an important cataract risk factor.

In other embodiments, the target condition capable of chemically modifying the cleavable linker is via exposure to nucleophilic or basic reagents, electrophilic or acidic reagents, reducing reagents, oxidizing reagents, or an organometallic compound.

Detection of a target condition capable of chemically modifying a cleavable linker has many uses, including detecting processes that signal changes in toxicology, ground water contamination, or for biohazard or biotoxin detection.

In one embodiment, the target condition capable of chemically modifying the cleavable linker is an acidic pH. It is well known that local acidic conditions are correlated with various diseased states such as tumors, ischemia, and inflammation. More specifically, in tumor tissue, acidic extracellular pH is a result of anaerobic glycolysis from rapidly dividing tumor cells, and is a major hallmark of the tumor microenvironment.

Nanopore Devices

A nanopore device, as provided, includes at least a pore that forms an opening in a structure separating an interior space of the device into two volumes, and at least a sensor configured to identify objects (for example, by detecting changes in parameters indicative of objects) passing through the pore. Nanopore devices used for the methods described herein are also disclosed in PCT Publication WO/2013/012881, incorporated by reference in entirety.

The pore(s) in the nanopore device are of a nano scale or micro scale. In one aspect, each pore has a size that allows a small or large molecule or microorganism to pass. In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, the pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the nanopore device further includes means to move a polymer scaffold across the pore and/or means to identify objects that pass through the pore. Further details are provided below, described in the context of a two-pore device.

Compared to a single-pore nanopore device, a two-pore device can be more easily configured to provide good control of speed and direction of the movement of the polymer scaffold across the pores.

In one embodiment, the nanopore device includes a plurality of chambers, each chamber in communication with an adjacent chamber through at least one pore. Among these pores, two pores, namely a first pore and a second pore, are placed so as to allow at least a portion of a polymer scaffold to move out of the first pore and into the second pore. Further, the device includes a sensor at each pore capable of identifying the polymer scaffold during the movement. In one aspect, the identification entails identifying individual components of the polymer scaffold. In another aspect, the identification entails identifying fusion:payload molecules bound to the polymer scaffold. When a single sensor is employed, the single sensor may include two electrodes placed at both ends of a pore to measure an ionic current across the pore. In another embodiment, the single sensor comprises a component other than electrodes.

In one aspect, the device includes three chambers connected through two pores. Devices with more than three chambers can be readily designed to include one or more additional chambers on either side of a three-chamber device, or between any two of the three chambers. Likewise, more than two pores can be included in the device to connect the chambers.

In one aspect, there can be two or more pores between two adjacent chambers, to allow multiple polymer scaffolds to move from one chamber to the next simultaneously. Such a multi-pore design can enhance throughput of enzyme activity analysis in the device. For multiplexing, one chamber could have a cleavable linker for one target type, and another chamber could have a different cleavable linker for another target type, with sample being exposed to all chambers prior to nanopore sensing.

In some aspects, the device further includes means to move a polymer scaffold from one chamber to another. In one aspect, the movement results in loading the polymer scaffold across both the first pore and the second pore at the same time. In another aspect, the means further enables the movement of the polymer scaffold, through both pores, in the same direction.

For instance, in a three-chamber two-pore device (a “two-pore” device), each of the chambers can contain an electrode for connecting to a power supply so that a separate voltage can be applied across each of the pores between the chambers.

In accordance with one embodiment of the present disclosure, provided is a device comprising an upper chamber, a middle chamber and a lower chamber, wherein the upper chamber is in communication with the middle chamber through a first pore, and the middle chamber is in communication with the lower chamber through a second pore. Such a device may have any of the dimensions or other characteristics previously disclosed in U.S. Publ. No. 2013-0233709, entitled Dual-Pore Device, which is herein incorporated by reference in its entirety.

In one aspect, each pore is at least about 1 nm in diameter. Alternatively, each pore is at least about 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 60 nm, 70 nm, 80 nm, 90 nm, or 100 nm in diameter.

In one aspect, each pore is no more than about 100 nm in diameter. Alternatively, the pore is no more than about 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, or 10 nm in diameter.

In one aspect, the pore has a diameter that is between about 1 nm and about 100 nm, or alternatively between about 2 nm and about 80 nm, or between about 3 nm and about 70 nm, or between about 4 nm and about 60 nm, or between about 5 nm and about 50 nm, or between about 10 nm and about 40 nm, or between about 15 nm and about 30 nm.

In some aspects, the pore has a substantially round shape. “Substantially round”, as used here, refers to a shape that is at least about 80 or 90% in the form of a cylinder. In some embodiments, the pore is square, rectangular, triangular, oval, or hexangular in shape.

In one aspect, the pore has a depth that is between about 1 nm and about 10,000 nm, or alternatively, between about 2 nm and about 9,000 nm, or between about 3 nm and about 8,000 nm, etc.

In some aspects, the nanopore extends through a membrane. For example, the pore may be a protein channel inserted in a lipid bilayer membrane or it may be engineered by drilling, etching, or otherwise forming the pore through a solid-state substrate such as silicon dioxide, silicon nitride, grapheme, or layers formed of combinations of these or other materials. Nanopores are sized to permit passage through the pore of the scaffold:fusion:payload, or the product of this molecule following enzyme activity. In other embodiments, temporary blockage of the pore may be desirable for discrimination of molecule types.

In some aspects, the length or depth of the nanopore is sufficiently large so as to form a channel connecting two otherwise separate volumes. In some such aspects, the depth of each pore is greater than 100 nm, 200 nm, 300 nm, 400 nm, 500 nm, 600 nm, 700 nm, 800 nm, or 900 nm. In some aspects, the depth of each pore is no more than 2000 nm or 1000 nm.

In one aspect, the pores are spaced apart at a distance that is between about 10 nm and about 1000 nm. In some aspects, the distance between the pores is greater than 1000 nm, 2000 nm, 3000 nm, 4000 nm, 5000 nm, 6000 nm, 7000 nm, 8000 nm, or 9000 nm. In some aspects, the pores are spaced no more than 30000 nm, 20000 nm, or 10000 nm apart. In one aspect, the distance is at least about 10 nm, or alternatively, at least about 20 nm, 30 nm, 40 nm, 50 nm, 60 nm, 70 nm, 80 nm, 90 nm, 100 nm, 150 nm, 200 nm, 250 nm, or 300 nm. In another aspect, the distance is no more than about 1000 nm, 900 nm, 800 nm, 700 nm, 600 nm, 500 nm, 400 nm, 300 nm, 250 nm, 200 nm, 150 nm, or 100 nm.

In yet another aspect, the distance between the pores is between about 20 nm and about 800 nm, between about 30 nm and about 700 nm, between about 40 nm and about 500 nm, or between about 50 nm and about 300 nm.

The two pores can be arranged in any position so long as they allow fluid communication between the chambers and have the prescribed size and distance between them. In one aspect, the pores are placed so that there is no direct blockage between them. Still, in one aspect, the pores are substantially coaxial.

In one aspect, the device has electrodes in the chambers connected to one or more power supplies. In some aspects, the power supply includes a voltage-clamp or a patch-clamp, which can supply a voltage across each pore and measure the current through each pore independently. In this respect, the power supply and the electrode configuration can set the middle chamber to a common ground for both power supplies. In one aspect, the power supply or supplies are configured to apply a first voltage V₁ between the upper chamber (Chamber A) and the middle chamber (Chamber B), and a second voltage V₂ between the middle chamber and the lower chamber (Chamber C).

In some aspects, the first voltage V₁ and the second voltage V₂ are independently adjustable. In one aspect, the middle chamber is adjusted to be a ground relative to the two voltages. In one aspect, the middle chamber comprises a medium for providing conductance between each of the pores and the electrode in the middle chamber. In one aspect, the middle chamber includes a medium for providing a resistance between each of the pores and the electrode in the middle chamber. Keeping such a resistance sufficiently small relative to the nanopore resistances is useful for decoupling the two voltages and currents across the pores, which is helpful for the independent adjustment of the voltages.

Adjustment of the voltages can be used to control the movement of charged particles in the chambers. For instance, when both voltages are set in the same polarity, a properly charged particle can be moved from the upper chamber to the middle chamber and to the lower chamber, or the other way around, sequentially. In some aspects, when the two voltages are set to opposite polarity, a charged particle can be moved from either the upper or the lower chamber to the middle chamber and kept there.

The adjustment of the voltages in the device can be particularly useful for controlling the movement of a large molecule, such as a charged polymer scaffold, that is long enough to cross both pores at the same time. In such an aspect, the direction and the speed of the movement of the molecule can be controlled by the relative magnitude and polarity of the voltages as described below.

The device can contain materials suitable for holding liquid samples, in particular, biological samples, and/or materials suitable for nanofabrication. In one aspect, such materials include dielectric materials such as, but not limited to, silicon, silicon nitride, silicon dioxide, graphene, carbon nanotubes, TiO₂, HfO₂, Al₂O₃, or other metallic layers, or any combination of these materials. In some aspects, for example, a single sheet of graphene membrane of about 0.3 nm thick can be used as the pore-bearing membrane.

Devices that are microfluidic and that house two-pore microfluidic chip implementations can be made by a variety of means and methods. For a microfluidic chip comprised of two parallel membranes, both membranes can be simultaneously drilled by a single beam to form two concentric pores, though using different beams on each side of the membranes is also possible in concert with any suitable alignment technique. In general terms, the housing ensures sealed separation of Chambers A-C.

In one aspect, the device includes a microfluidic chip (labeled as “Dual-pore chip”) is comprised of two parallel membranes connected by spacers. Each membrane contains a pore drilled by a single beam through the center of the membrane. Further, the device preferably has a Teflon® housing or polycarbonate housing for the chip. The housing ensures sealed separation of Chambers A-C and provides minimal access resistance for the electrode to ensure that each voltage is applied principally across each pore.

More specifically, the pore-bearing membranes can be made with transmission electron microscopy (TEM) grids with a 5-100 nm thick silicon, silicon nitride, or silicon dioxide windows. Spacers can be used to separate the membranes, using an insulator, such as SU-8, photoresist, PECVD oxide, ALD oxide, ALD alumina, or an evaporated metal material, such as Ag, Au, or Pt, and occupying a small volume within the otherwise aqueous portion of Chamber B between the membranes. A holder is seated in an aqueous bath that is comprised of the largest volumetric fraction of Chamber B. Chambers A and C are accessible by larger diameter channels (for low access resistance) that lead to the membrane seals.

A focused electron or ion beam can be used to drill pores through the membranes, naturally aligning them. The pores can also be sculpted (shrunk) to smaller sizes by applying a correct beam focusing to each layer. Any single nanopore drilling method can also be used to drill the pair of pores in the two membranes, with consideration to the drill depth possible for a given method and the thickness of the membranes. Predrilling a micro-pore to a prescribed depth and then a nanopore through the remainder of the membranes is also possible to further refine the membrane thickness.

By virtue of the voltages present at the pores of the device, charged molecules can be moved through the pores between chambers. Speed and direction of the movement can be controlled by the magnitude and polarity of the voltages. Further, because each of the two voltages can be independently adjusted, the direction and speed of the movement of a charged molecule can be finely controlled in each chamber.

One example concerns a charged polymer scaffold, such as a DNA, having a length that is longer than the combined distance that includes the depth of both pores plus the distance between the two pores. For example, a 1000 by dsDNA is about 340 nm in length, and would be substantially longer than the 40 nm spanned by two 10 nm-deep pores separated by 20 nm. In a first step, the polynucleotide is loaded into either the upper or the lower chamber. By virtue of its negative charge under a physiological condition at a pH of about 7.4, the polynucleotide can be moved across a pore on which a voltage is applied. Therefore, in a second step, two voltages, in the same polarity and at the same or similar magnitudes, are applied to the pores to move the polynucleotide across both pores sequentially.

At about the time when the polynucleotide reaches the second pore, one or both of the voltages can be changed. Since the distance between the two pores is selected to be shorter than the length of the polynucleotide, when the polynucleotide reaches the second pore, it is also in the first pore. A prompt change of polarity of the voltage at the first pore, therefore, will generate a force that pulls the polynucleotide away from the second pore.

Assuming that the two pores have identical voltage-force influence and |V₁|=|V₂|+δV, the value δV>0 (or <0) can be adjusted for tunable motion in the |V₁| (or V₂) direction. In practice, although the voltage-induced force at each pore will not be identical with V₁=V₂, calibration experiments can identify the appropriate bias voltage that will result in equal pulling forces for a given two-pore chip; and variations around that bias voltage can then be used for directional control.

If, at this point, the magnitude of the voltage-induced force at the first pore is less than that of the voltage-induced force at the second pore, then the polynucleotide will continue crossing both pores towards the second pore, but at a lower speed. In this respect, it is readily appreciated that the speed and direction of the movement of the polynucleotide can be controlled by the polarities and magnitudes of both voltages. As will be further described below, such a fine control of movement has broad applications. For quantitating activity of enzymes, the utility of two-pore device implementations is that during controlled delivery and sensing, the modification or cleavage of the cleavable linker can be repeatedly measured, to add confidence to the detection result. Additionally, more than one fusion:payload could be added at distinct sites along the scaffold, to detect the activity of more than one enzyme at a time (multiplexing).

Accordingly, in one aspect, provided is a method for controlling the movement of a charged polymer scaffold through a nanopore device. The method comprises loading a sample comprising a charged polymer scaffold in one of the upper chamber, middle chamber or lower chamber of the device of any of the above embodiments, wherein the device is connected to one or more power supplies for providing a first voltage between the upper chamber and the middle chamber, and a second voltage between the middle chamber and the lower chamber; setting an initial first voltage and an initial second voltage so that the polymer scaffold moves between the chambers, thereby locating the polymer scaffold across both the first and second pores; and adjusting the first voltage and the second voltage so that both voltages generate force to pull the charged polymer scaffold away from the middle chamber (voltage-competition mode), wherein the two voltages are different in magnitude, under controlled conditions, so that the charged polymer scaffold moves across both pores in either direction and in a controlled manner.

In one aspect, the sample containing the charged polymer scaffold is loaded into the upper chamber and the initial first voltage is set to pull the charged polymer scaffold from the upper chamber to the middle chamber and the initial second voltage is set to pull the polymer scaffold from the middle chamber to the lower chamber. Likewise, the sample can be initially loaded into the lower chamber, and the charged polymer scaffold can be pulled to the middle and the upper chambers.

In another aspect, the sample containing the charged polymer scaffold is loaded into the middle chamber; the initial first voltage is set to pull the charged polymer scaffold from the middle chamber to the upper chamber; and the initial second voltage is set to pull the charged polymer scaffold from the middle chamber to the lower chamber.

In one aspect, real-time or on-line adjustments to the first voltage and the second voltage at step (c) are performed by active control or feedback control using dedicated hardware and software, at clock rates up to hundreds of megahertz. Automated control of the first or second or both voltages is based on feedback of the first or second or both ionic current measurements.

Sensors

As discussed above, in various aspects, the nanopore device further includes one or more sensors to carry out the detection of the activity status of the target molecule (e.g., enzyme).

The sensors used in the device can be any sensor suitable for identifying cleavage of the cleavable linker by the target molecule or target condition. For instance, a sensor can be configured to identify the polymer (e.g., a polymer scaffold) by measuring a current, a voltage, a pH value, an optical feature, or residence time associated with the polymer. In other aspects, the sensor may be configured to identify one or more individual components of the polymer or one or more components bound or attached to the polymer. The sensor may be formed of any component configured to detect a change in a measurable parameter where the change is indicative of the polymer, a component of the polymer, or preferably, a component bound or attached to the polymer. In one aspect, the sensor includes a pair of electrodes placed at two sides of a pore to measure an ionic current across the pore when a molecule or other entity, in particular a polymer scaffold, moves through the pore. In certain aspects, the ionic current across the pore changes measurably when a polymer scaffold segment passing through the pore is bound to a fusion:payload molecule. Such changes in current may vary in predictable, measurable ways corresponding with, for example, the presence, absence, and/or size of the fusion:payload molecule present.

In a preferred embodiment, the sensor comprises electrodes that apply voltage and are used to measure current across the nanopore. Translocations of molecules through the nanopore provides electrical impedance (Z) which affects current through the nanopore according to Ohm's Law, V=IZ, where V is voltage applied, I is current through the nanopore, and Z is impedance. Inversely, the conductance G=1/Z are monitored to signal and quantitate nanopore events. The result when a molecule translocates through a nanopore in an electrical field (e.g., under an applied voltage) is a current signature that may be correlated to the molecule passing through the nanopore upon further analysis of the current signal.

When residence time measurements from the current signature are used, the size of the component can be correlated to the specific component based on the length of time it takes to pass through the sensing device.

In one embodiment, a sensor is provided in the nanopore device that measures an optical feature of the polymer, a component (or unit) of the polymer, or a component bound or attached to the polymer. One example of such measurement includes the identification of an absorption band unique to a particular unit by infrared (or ultraviolet) spectroscopy.

In some embodiments, the sensor is an electric sensor. In some embodiments, the sensor detects a fluorescent signature. A radiation source at the outlet of the pore can be used to detect that signature.

Discrimination from Background

In some aspects, the target molecule present in the sample can be from original (even filtered) natural fluids (blood, saliva, urine, etc.), which have a vast population of background molecules. Such background molecules, when sufficiently negatively charged with a positive applied voltage, and pass through the nanopore. In some cases, such nanopore events may appear to look like the scaffold:fusion:payload construct or the products (scaffold, payload) following cleavage of the cleavable linker. As such, these background molecules can produce false positives, generating a high error rate of detection. Adding sufficient sample prep to remove larger molecules will help this, but background molecules that create false positive events will still be present.

To provide discrimination between background molecules and scaffold molecules (with or without attachment of the fusion:payload), a scaffold-labeling scheme can be used. Scaffold labeling schemes are also disclosed in U.S. provisional application No. 61/993,985, incorporated by reference in entirety.

Specifically, a label or a sequence of labels are bound to the polymer scaffold to provide a unique current signature that can be used to identify the presence and/or identity of a polymer scaffold that has translocated through a nanopore. Within the same event signature, the presence or absence of the fusion:payload signal whether the cleavable linker was modified on that molecule.

In another embodiment, the length of the scaffold alone provides a discriminatory signature that is sufficient distinct from background, while also preserving discriminatory power between scaffold:fusion:payload and scaffold alone (following cleavage of the cleavable linker).

Assigning Statistical Significance to Detection

In some embodiments, aggregating the set of sensor measurements recorded over time and applying mathematical tools are performed to assign a numerical confidence value to detection of the target molecule or condition suspected to be present in a sample, as detailed in the previous section.

A quantitative method of discriminating a molecule type from background (i.e., other molecule types) based on differences in nanopore event population characteristics was recently developed (Morin, T. J., et al., “Nanopore-based target sequence detection,” submitted to PloS One, Dec. 31, 2015). This method of discrimination means a specific molecule type can be detected among the presence of varying types of other background molecules, and that the statistical significance of detection can be assigned (e.g., detection of reagent X with 99% confidence). To apply the method to the examples provided below, we first summarize the method here.

In general terms, there are two categories of molecules in the chamber above the pore: type 1 are all the background molecules, and type 2 are the molecules of interest. In Example 3 below, for example, DNA-payload could be considered as the type 2 molecules, with DNA alone being considered as background (type 1). Based on data from experiments, we identify an event signature criterion that is present in a significant fraction of type 2 events, and present in a relatively smaller fraction of type 1 events. An event is “tagged” as being type 2 if the signature criterion is met for that event. A signature could depend on 6G, duration, the number and characteristics of levels within each event, and/or any other numeric value or combination of values computed from the event signal. We define p as the probability that a capture event is type 2. In control experiments without type 2 molecules p=0, and in experiments with type 2 molecules p >0 but its value is not known. We define the false positive probability q1=Pr(tagged|type 1 event). In a control experiment or set of experiments without type 2 molecules, q1 is determined with good accuracy from a large number of capture events. In a detection experiment to determine if type 2 molecules are present in bulk solution, the probability that a capture event is tagged is a function of p and can be approximated as:

Q(p)=(Number of tagged events)/N

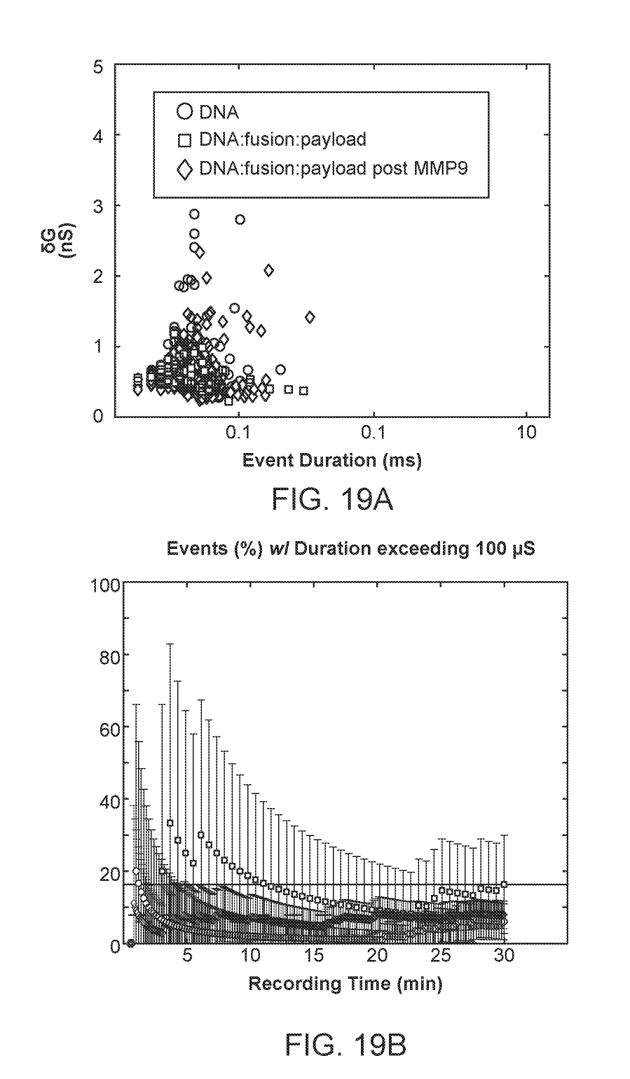

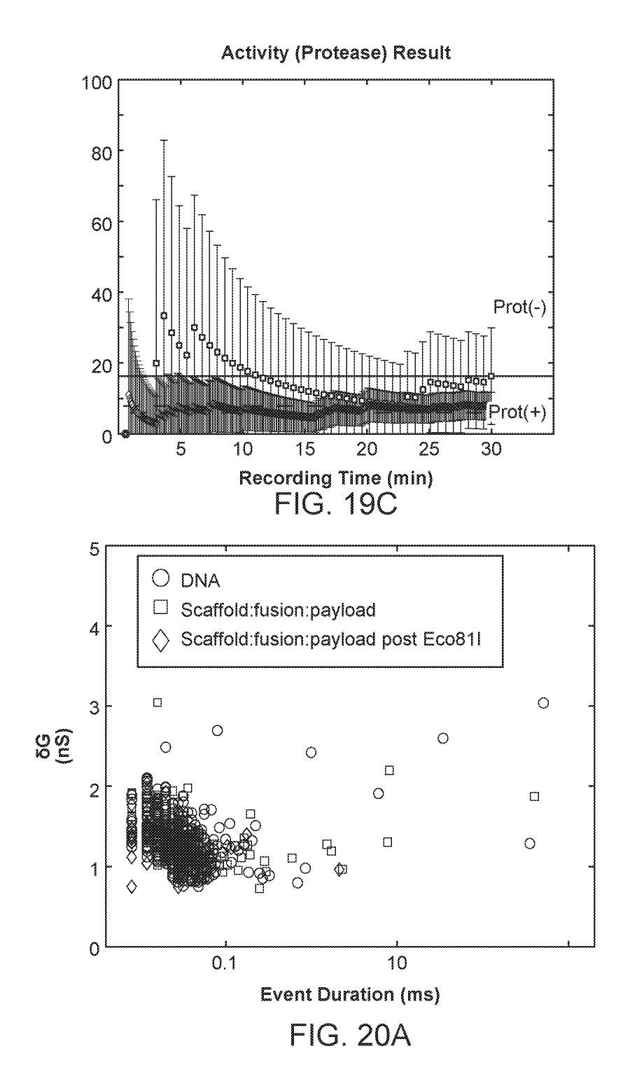

In the formula, N is the total number of events. The 99% confidence interval Q(p) Q_{sd}(p) can be computed with Q_{sd}(p)=2.57*sqrt{Q(p)*(1−Q(p))/N}, with sqrt{ } the square root function. During the course of an experiment, the value for Q(p) converges and the uncertainty bounds attenuate as the number of events N increases. A plot of Q(p)±Q_{sd} (p) as a function of recording time shows how it evolves for each reagent type (FIG. 19b for Example 3). In a control experiment without type 2 molecules, observe that Q(0)=q1. In a control experiment with type 2 molecules known to be present at some probability p*>0, the computed value Q(p*) can be used in a detection experiment to determine if type 2 molecules are absent, as defined below.

In a detection experiment, type 2 molecules are present with 99% confidence when the following criteria is true:

Q(p)−Q_{sd}(p)>q1 (1.)

If the criteria above is true, we conclude p >0; if it is untrue, we cannot say p>0. In a detection experiment, type 2 molecules are absent with 99% confidence when the following criteria is true:

Q(p)+Q_{sd}(p)<Q(p*) (2.)

If the criteria above is true, we conclude that p=0; otherwise, we cannot make a conclusion. The framework is utilized in the Examples provided below.

Estimating Target Molecule Concentration

In some embodiments, aggregating the set of sensor measurements recorded over time and applying mathematical tools are performed to estimate the concentration of the target molecule or condition suspected to be present in a sample.

In some embodiments, the process (incubate sample with scaffold:fusion:payload reagent and perform nanopore experiments) can be repeated while varying concentration of one or more of the scaffold, fusion, payload and/or target molecule or condition suspected to be present in a sample. The data sets can then be combine to glean more information. In one embodiment, the total concentration of active enzyme is to be estimated by applying mathematical tools to the aggregated data sets.

Following methods in the literature (Wang, Hongyun, et al., “Measuring and Modeling the Kinetics of Individual DNA-DNA Polymerase Complexes on a Nanopore.” ACS Nano 7, no. 5 (May 28, 2013): 3876-86. doi:10.1021/nn401180j; Benner, Seico, et al., “Sequence-Specific Detection of Individual DNA Polymerase Complexes in Real Time Using a Nanopore.” Nature Nanotechnology 2, no. 11 (Oct. 28, 2007): 718-24 (doi:10.1038/nnano.2007.344), one can apply biophysical models to nanopore data to quantitate the binding, bond-breakage and subsequent dissociation kinetics between the target enzyme and its substrate (e.g., a cleavable linker or cleavable domain).

In our assays, the nanopore is sampling and measuring individual molecules from the bulk-phase. In the presence of a target molecule, the cleavable linker within the scaffold:fusion:payload will be modified (e.g., cleaved) at some rate that is proportional to the concentration of the target. At high target concentrations relative to the scaffold:fusion:payload concentration, cleavage will proceed rapidly, and all of the cleavable linkers will be cleaved, resulting in detection of only scaffold and payload molecules, and any other background molecules. At lower concentrations relative to the scaffold:fusion:payload concentration, cleavage will proceed more slowly, and within a 10 minutes recording period a majority of the scaffold events will signal scaffold:fusion:payload intact passing through the pore. At intermediate concentrations relative to the scaffold:fusion:payload concentration, a non-zero percentage of scaffold events will be flagged as being in tact scaffold:fusion:payload, and this percentage will decrease over time as the reaction progresses to completion.

To estimate total active enzyme concentration, a repeated experiment can be conducted with a nanopore and using a different concentration of scaffold:fusion:payload reagent each time, from low (1 pM) to high (100 nM), with the target molecule concentration being conserved by using a portion of a common sample. By measuring the time evolution of the percentage of scaffold events flagged as being in tact scaffold:fusion:payload, a modeling framework similar to those in cited work can be used to quantitate total enzyme concentration. Specifically, time-dependent measurements were used in Wang, Hongyun, et al., “Measuring and Modeling the Kinetics of Individual DNA-DNA Polymerase Complexes on a Nanopore.” ACS Nano 7, no. 5 (May 28, 2013): 3876-86. doi:10.1021/nn401180j, with a model that explicitly allows estimation of the total enzyme concentration.

To estimate total active enzyme concentration, a multi-nanopore array can be implemented. Each nanopore will measured a different concentration of scaffold:fusion:payload reagents, from low (1 pM) to high (100 nM). By measuring the time evolution of the percentage of scaffold events flagged as being in tact scaffold:fusion:payload at each nanopore in parallel, a modeling framework similar to those in cited work can be used to quantitate total enzyme concentration.

### EXAMPLES

The present technology is further defined by reference to the following example and experiments. It will be apparent to those skilled in the art that many modifications may be practiced without departing from the scope of the current invention.

### Example 1: Nanopore Detection of DNA Scaffold

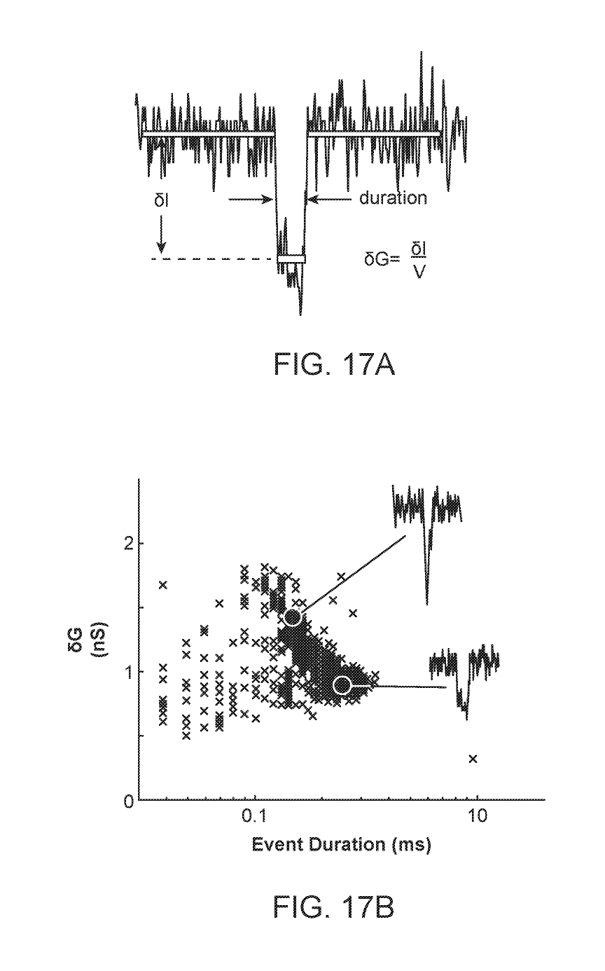

A solid-state nanopore is a nano-scale opening formed in a thin solid-state membrane that separates two aqueous volumes. A voltage-clamp amplifier applies a voltage V across the membrane while measuring the ionic current through the open pore. Unlike any other single-molecule sensor, the nanopore device can be packaged into a hand-held form factor at very low cost. When a single charged molecule such as a double-stranded DNA (dsDNA) is captured and driven through the pore by electrophoresis, the measured current shifts, and the conductance shift depth (δG=δI/V) and duration are used to characterize the event (FIG. 17a).

After recording many events during an experiment, distributions of the events are analyzed to characterize the corresponding molecule. FIG. 17b shows the event characteristics for 3.2 kb dsDNA passing through an 27 nm diameter nanopore at voltage V=100 mV (1M LiCl). The two encircled representative events show: a wider and shallower event corresponding to the DNA passing through unfolded; and a faster but deeper event corresponding to the DNA passing through folded. For dsDNA that is −1 kb and shorter, the DNA passes through the pore only in an unfolded state.

### Example 2: A Scaffold:Fusion:Payload Containing a Cleavable Linker for a Protease and a Cleavable Linker for an Endonuclease

For the purpose of demonstrating our assay experimentally, we designed and built a single construct that comprises two distinct cleavable linkers within a single fusion molecule, as depicted in FIG. 12. With this single construct, we sought to demonstrate detection of activity of an endonuclease, and separately to demonstrate detection of activity of a protease.

A DNA scaffold was generated using a dibenzocylcooctyne (DBCO) modified primer, effectively labeling the molecule with a DBCO chemical group to be used for conjugation purposes via copper-free “click” chemistry (FIG. 6). The PCR template included the endonuclease sensitive sequence, CC/T(N)AGG (/represents cleavage site, N represents any DNA nucleobase C, G, T or A). In the endonuclease activity assay, a portion of the fusion molecule then comprises the target DNA sequence (FIG. 12A). This modified DNA scaffold was subsequently allowed to incubate overnight at 37° C. with 1000-fold excess of an azide-tagged molecule containing the peptide sequence SGKGPRQITA (0.01M sodium phosphate+300 mM NaCl, pH 7.4). This peptide had previously been isolated from a phage display library and identified as highly sensitive to MMP9 activity (Kridel, Steven J., et al. “Substrate hydrolysis by matrix metalloproteinase-9. Journal of Biological Chemistry 276.23 (2001): 20572-20578). In the protease MMP9 activity assay, a portion of the fusion molecule then comprises the target peptide sequence (FIG. 12A). The scaffold:fusion:payload molecule consisted of (from N-terminus to C-terminus): DNA scaffold, DNA fusion (containing endonuclease sequence cleavable linker to the end of the DNA), an azide chemical handle, PEG₄, a flexible Gly-Ser motif, MMP9-sensitive peptide sequence SGKGPRQITA, flexible Gly-Ser motif, Cys-5 kDa PEG, and biotin (FIG. 12A, synthesized by Bio-Synthesis, Inc., Lewisville, Tex.).

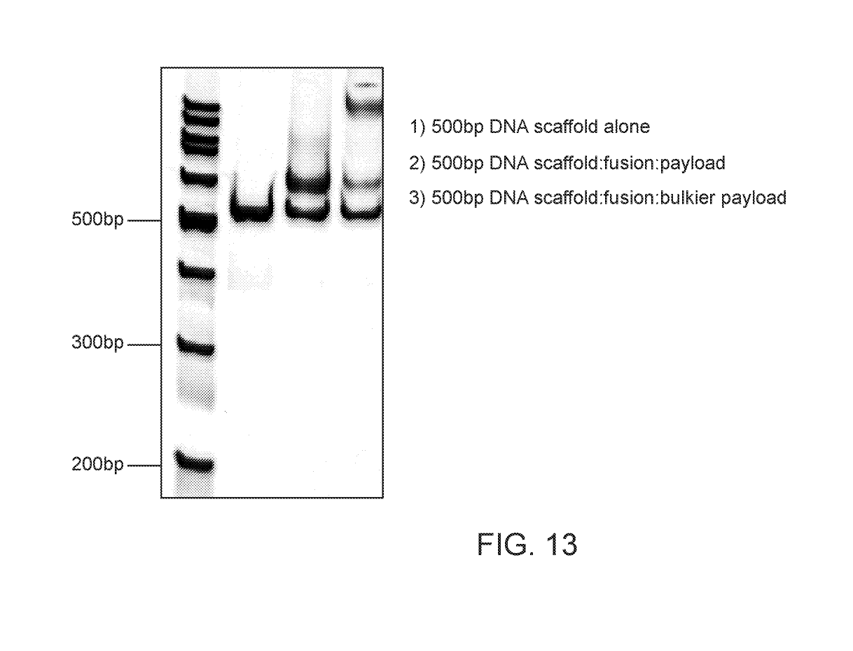

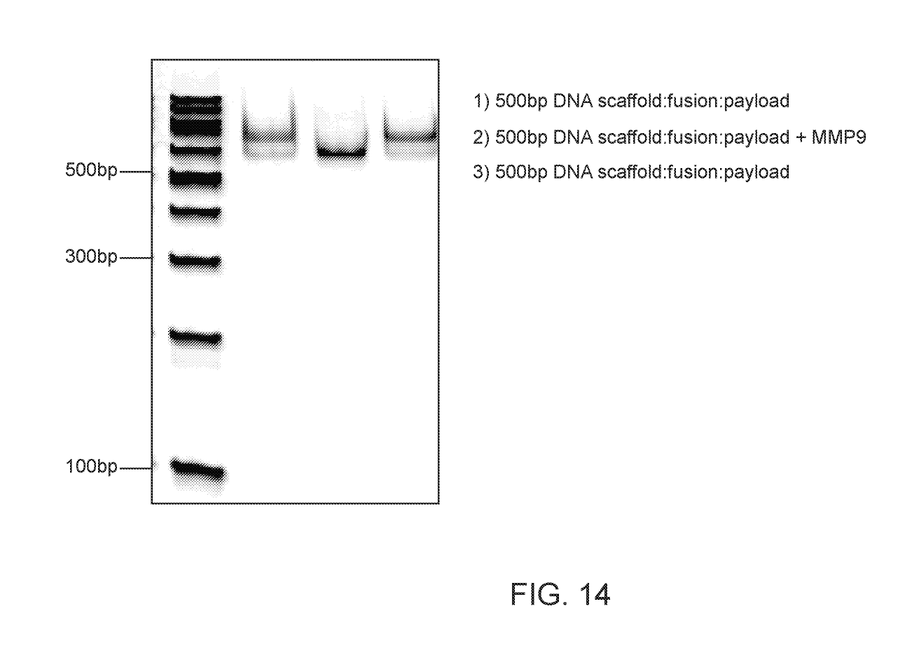

Successful linking of the payload molecule as indicated in FIG. 12A was verified via an EMSA gel stained with the DNA-specific dye Sybr Green (FIG. 13, Lane 2, upper band). Conjugation of the DNA scaffold to the fusion:payload molecule including the sensitive cleavable linker resulted in ˜50% of the desired product (FIG. 13, Lane 2, upper band). To purify pure conjugated construct away from unconjugated DNA alone, the product was gel-extracted from the polyacrylamide gel and resuspended in enzyme activity buffer per the manufacturer's instructions (the result of this process, pure DNA-payload is shown in FIG. 14, Lanes 1 and 3).

### Example 3: Nanopore Detection and Discrimination of DNA, DNA-Payload and DNA-Payload-Monostreptavidin

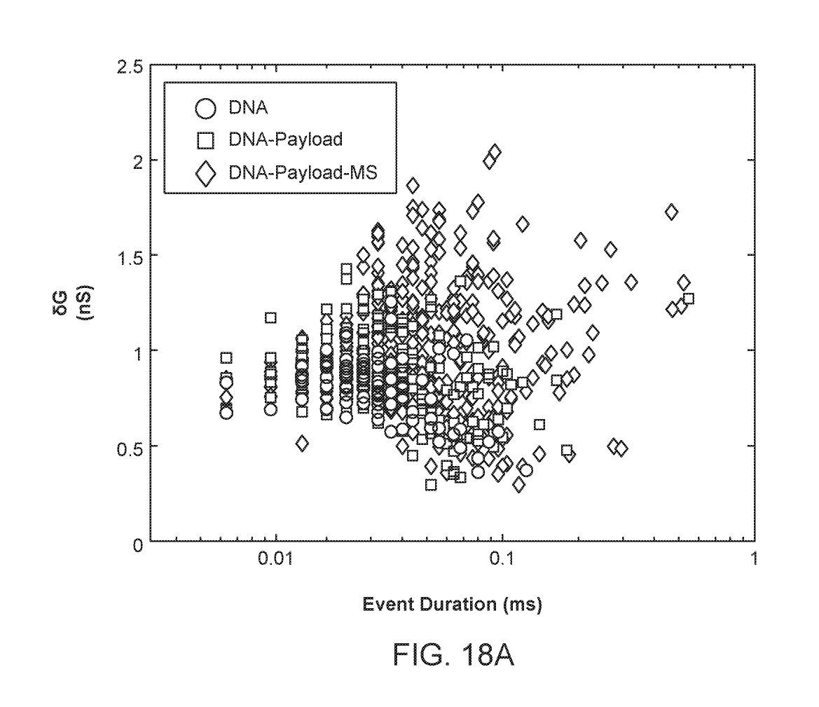

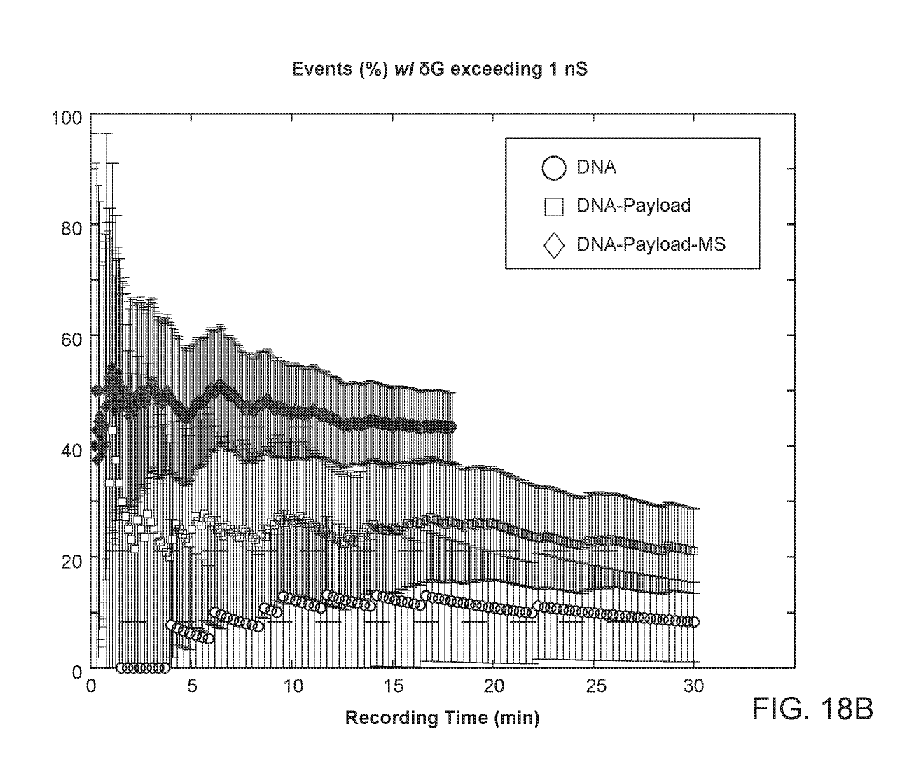

A 500 bp DNA scaffold alone was measured with a 15 nm nanopore (0.2 nM, 100 mV, 1M LiCl, 10 mM Tris, 1 mM EDTA, pH 8.0), producing 97 events in 30 minutes (FIG. 18a). Few events (8.3%) hit a depth of at least 1 nS (FIG. 18b). Following removal of the DNA from the chamber adjacent to the nanopore, 0.2 nM DNA-payload reagent was added, where DNA-payload here refers to the complex referenced in Example 2 and FIG. 12. The DNA-payload reagent produced 190 events over 30 minutes, with an increase to 21.1% of events hitting a depth of at least 1 nS (FIG. 18b). Following removal of the DNA-payload reagent, 0.2 nM DNA-payload that had been incubated with monostreptavidin (FIG. 13, Lane 3, upper band) was added to the chamber for nanopore measurement, where monostreptavidin binds to the free biotin at the end of the payload (as shown in FIG. 6B). By adding monostreptavidin, the increased size of each DNA-payload-monostreptavidin molecule resulted in an increase in event depth and duration for a majority of the 414 events recorded over 18 minutes (FIG. 18a). The population increased to 43.5% of events hitting a depth of at least 1 nS (FIG. 18b).

The visual shift in event populations (FIG. 18a) is consistent with the increase in size of the molecule, from DNA to DNA-payload, and then to DNA-payload-monostreptavidin. Our quantitative method for detecting a specific molecule type among the presence of varying types of other background molecules can be applied to these data, so that the statistical significance of detection can be assigned.

If DNA is considered type 1 and DNA-payload considered type 2, an example criteria is to tag an event as type 2 if δG>1 nS. The DNA alone population can be used to compute q1=0.082 (8.2%). The DNA-payload experiment can be used as a mock detection experiment and to determine if type 2 molecules are present by applying equation (1) of the mathematical framework. The result is 0.211−0.076=0.134>0.082, which means we can say that type 2 (DNA-payload) molecules are present with 99% confidence.

Next, DNA and DNA-payload are considered type 1 and DNA-payload-monostreptavidin is considered type 2, and we can use the same criteria (δG>1 nS) to tag an event as type 2. The DNA alone and DNA-payload populations can be used to establish q1=0.211 (using the larger of the two values 0.082 and 0.211, as a viable false positive probability). As before, the DNA-payload-monostreptavidin population can be used as a mock detection experiment, and we test if type 2 molecules by applying equation (1). The result is 0.435−0.063=0.372>0.211, which means we can say that type 2 (DNA-payload-monostreptavidin) molecules are present with 99% confidence.

Keeping DNA-payload-monostreptavidin as the type 2 molecule of interest, we can also examine a mock complementary test in which we apply equation (2) of the mathematical framework. Specifically, we can consider the DNA-payload data as an “unknown” reagent from which we want to know if the bulkier DNA-payload-monostreptavidin is absent. We again use the criteria (δG>1 nS) to tag an event as type 2. From the DNA-payload-monostreptavidin control experiment, we have Q(p*)=0.435. From the “unknown” (DNA-payload) data and applying equation (2), the result is 0.211+0.076=0.287<0.435, which means we can say with 99% confidence that type 2 (DNA-payload-monostreptavidin) molecules are not present in the mock “unknown” reagent (DNA-payload, sans monostreptavidin).

### Example 4: Digestion of an MMP9 Sensitive Molecular Construct Followed by Nanopore Detection

Matrix-metalloproteinase 9 (MMP9) is a 92 kDa extracellular matrix-degrading enzyme (ECM) that has been found to be involved in a wide variety of normal human physiological processes. Timely degradation of the ECM is an important feature of tissue repair, morphogenesis, and development. Because of its critical role in normal human physiology, aberrant expression and/or activity of MMP9 has been associated with a number of serious human medical conditions including but not limited to cardiovascular disease, rheumatoid arthritis, and a variety of malignancies (Nagase, Hideaki, Robert Visse, and Gillian Murphy. “Structure and function of matrix metalloproteinases and TIMPs.” Cardiovascular research 69.3 (2006): 562-573). Considering this, MMP9 expression and proteolytic activity have been viewed as a valuable clinical diagnostic biomarker within the medical community.

The ability of MMP9 to cleave its target substrate SGKGPRQITA within a 300 bp or 500 bp DNA scaffold:fusion molecule:payload construct was first verified via an EMSA gel. The active catalytic subunit of MMP9 (39 kDa, Enzo Life Sciences) was allowed to incubate with DNA scaffold conjugated to the payload molecule in MMP9 activity buffer (50 mM Tris, 10 mM CaCl₂, 150 mM NaCl, 0.05% Brij 35, pH 7.5) at a 1:10 protease to substrate ratio overnight at 37° C. to ensure complete enzymatic degradation (FIG. 14, Lane 2). This incubation with MMP9 rendered a molecule that was more mobile in an acrylamide gel compared to the full construct (FIG. 14, Lanes 1 and 3), presumably due to complete enzymatic degradation of the construct in between the glutamine and isoleucine residues reported to be the cleavage site of the target substrate (Kridel, Steven J., et al. “Substrate hydrolysis by matrix metalloproteinase-9. Journal of Biological Chemistry 276.23 (2001): 20572-20578). Incubation of inactive MMP9 with the identical construct did not result in any shift between samples in the gel (data not shown), indicating that the change in electrophoretic mobility seen in Lane 2 is solely due to the proteolytic activity of the protease of interest, MMP9.

Next, we tested our method of nanopore detection of MMP9 cleaving its target substrate SGKGPRQITA within a 300 bp DNA:fusion:payload construct (referenced here as DNA-payload). First, 300 bp DNA scaffold alone at 0.4 nM was tested using a 15 nm diameter pore (100 mV, 1M LiCl), producing 146 events over 30 minutes with only 5.5% exceeding a duration of 0.1 ms (FIG. 19a,b). Subsequently, DNA-payload that had not been exposed to MMP9 activity was tested. This sample produced 49 events in 30 minutes, with 16.3% exceeding a duration of 0.1 ms (FIG. 19a,b). Next, following an incubation period between DNA-payload and MMP9 as previously described, the reaction mixture was tested on the pore at an equivalent DNA-payload concentration of 1.2 nM, producing 327 events over 30 minutes. If MMP9 degraded a sufficient majority of the substrate (i.e., the cleavable linker), the reaction mixture would contain DNA alone and payload alone, and as a result would have a reduction in the percentage of events exceeding 0.1 ms in duration compared to the un-degraded DNA-payload. This was in fact the case, with 7.6% of events exceeding 0.1 ms for DNA-payload post MMP9 degradation, a reduction from 16.3% for DNA-payload without degradation (FIG. 19a,b). A plot of Q(p)±Q_{sd}(p) as a function of recording time is shown for each reagent type (FIG. 19b).

We next implemented the method described previously for assigning statistical significance to nanopore detection assays. Specifically, with equation (2), we can implicitly detect MMP9 activity by testing for the absence of the DNA-payload molecular construct using the data generated by the reaction mixture between DNA-payload and MMP9. In this case, DNA-payload is the type 2 molecule to be detected, and a minimum event duration of 0.1 ms is chosen as the type 2 flagging criteria. In the control experiment with DNA-payload known to be present (without MMP9), we establish the value Q(p*)=0.163. Next, treating the DNA-payload post MMP9 activity as the “unknown” data, we apply equation (2). The result is 0.0765+0.038=0.117<0.163, which means we can say that type 2 (DNA-payload) molecules are absent with 99% confidence. As stated, the absence of DNA-payload implicitly shows that MMP9 degraded a sufficient percentage of molecules comprising its substrate. The MMP9 protease activity result applying equation (2) is displayed in FIG. 19c.

### Example 5: Digestion of an MMP9 Sensitive Molecular Construct in the Presence of Increasing Concentration of Urine

MMP9 has been found to be over-expressed and hyperactive in the urine of a number of human malignancies, including that of ovarian cancer (Coticchia, Christine M., et al. “Urinary MMP-2 and MMP-9 predict the presence of ovarian cancer in women with normal CA125 levels.” Gynecologic oncology 123.2 (2011): 295-300). For this reason, several commercially available kits (GE Healthcare, R&D Systems, Abcam) have been produced to analyze the concentration and/or activity levels of MMP9 that are present in human urine. In this example, the ability of MMP9 to degrade the scaffold:fusion:payload construct in the presence of an increasing concentration of urine was analyzed by an EMSA gel.

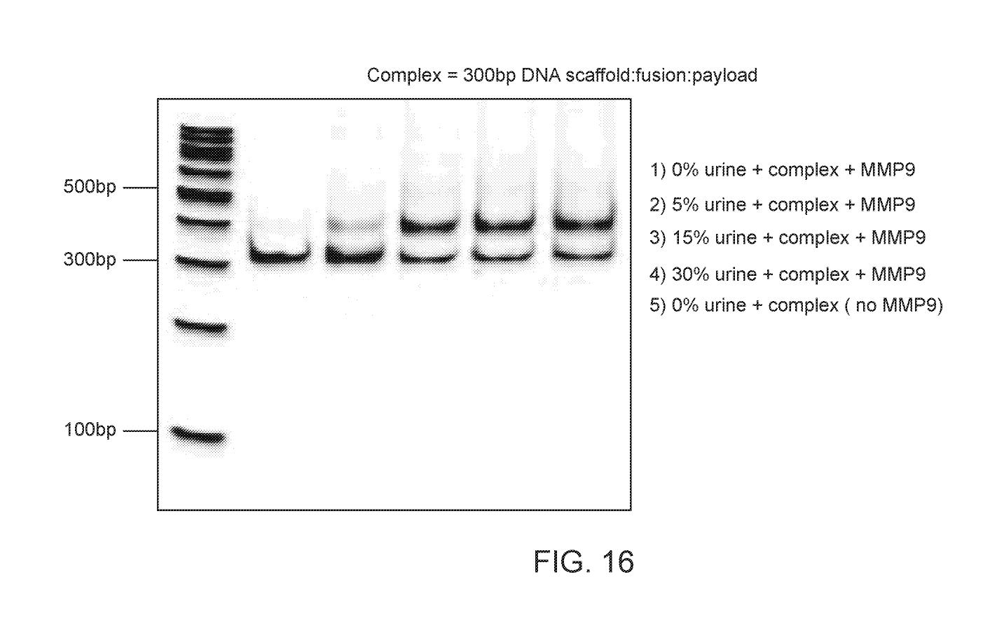

Conjugation of the cleavable linker:payload to a DBCO-modified DNA scaffold as described in Example 2 resulted in >75% final product (FIG. 16, Lane 5 upper band). This purified sample was then allowed to incubate with MMP9 in the presence of increasing amounts of human urine. In normal enzyme activity buffer, complete cleavage of the construct was observed through the disappearance of the upper conjugate band (Lane 1). As the concentration of urine was increased, it was found that complete enzyme inhibition occurred at >15% urine in solution (FIG. 16, Lanes 3 and 4), and moderate enzyme activity was detected in a solution of 5% urine (FIG. 16, Lane 2). The mechanism of protease inhibition was not investigated, but could be due to several factors including but not limited to pH change, the presence of native inhibitors in urine, or urea-mediated unfolding of the protein tertiary structure.

### Example 6: Hydrolysis of an Endonuclease Sensitive Construct Followed by Nanopore Detection

In a bacterial cell, restriction endonucleases act as a critical defense mechanism against the uptake of foreign DNA. Endonucleases recognize and degrade specific DNA sequences, protecting “self” while destroying potentially harmful foreign DNA such as would be the case in a virus infection. Staphylococcus aureus is a pathogenic bacteria that has been found to cause a wide variety of human infections which range from superficial skin lesions to severe systemic diseases. In this example, a DBCO-modified 500 bp DNA (comprising the scaffold and a portion of the fusion) is first created that includes the recognition sequence of the restriction enzyme Saul, CC/T(N)AGG (N represents C, G, T or A). Saul is an endonuclease that has been found to be present in all isolates of Staphylococcus aureus (Veiga, Helena, and Mariana G. Pinho. “Inactivation of the Saul type I restriction-modification system is not sufficient to generate Staphylococcus aureus strains capable of efficiently accepting foreign DNA.” Applied and environmental microbiology 75.10 (2009): 3034-3038). This DNA portion of the fusion is then conjugated to a payload molecule. Due to limited availability of Saul from commercial vendors, an endonuclease capable of hydrolytic cleavage at the same recognition sequence, Eco81I, was used.

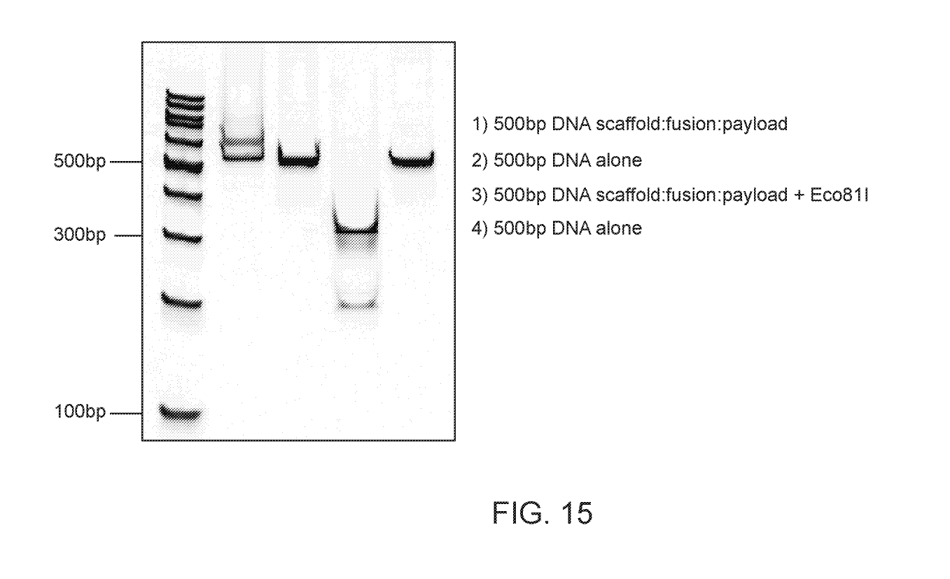

To assess the ability of Eco81I to degrade the engineered DNA scaffold:fusion:payload construct, the two were allowed to incubate together at 37° C. overnight to ensure complete DNA sequence-specific hydrolysis (20U Eco81I in 1× Tango Buffer, Thermo Scientific). Following incubation of the endonuclease with the scaffold:fusion:payload, an EMSA gel was ran to analyze the resulting products of the enzymatic reaction. A sample of conjugated DNA scaffold incubated without Eco81I (FIG. 15, Lane 1) displayed an upper band representative of intact construct, and a lower band of DNA that had not been conjugated to the payload molecule. However, when the sample in Lane 1 was allowed to incubate with Eco81I, complete degradation of DNA was observed (FIG. 15, Lane 3). Because the recognition sequence of Eco81I lies 194 bp away from the 3′ end of the DNA scaffold and is independent of the protease cleavable linker encoded in the payload molecule, both conjugated and unconjugated material (FIG. 15, Lane 1, upper and lower bands) was hydrolyzed by Eco81I. The expected products of 304 and 196 bp following the hydrolytic reaction of Eco81I on DNA are evident in Lane 3.

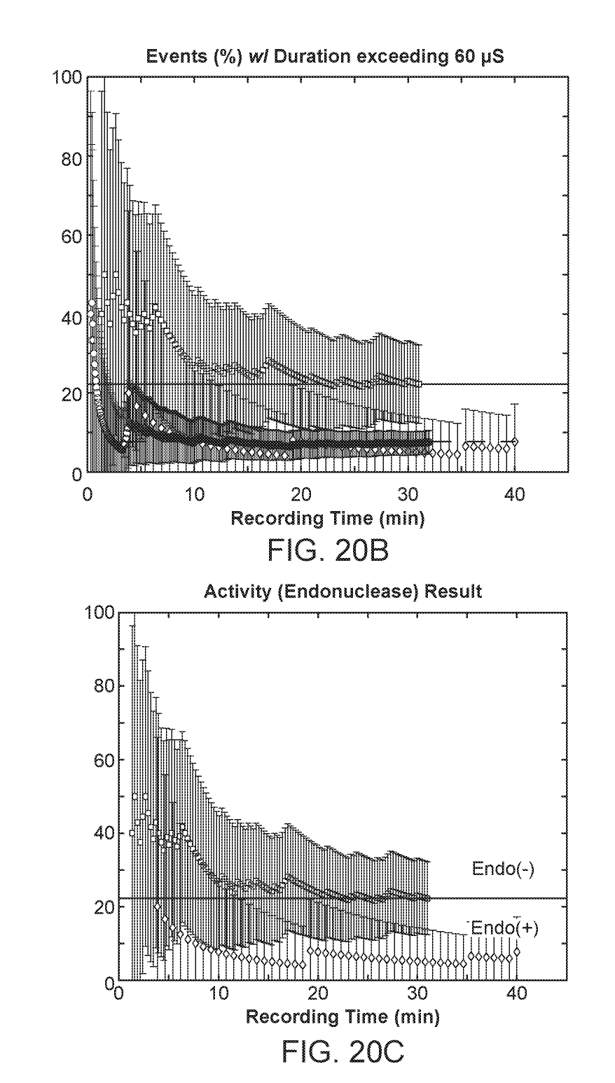

Following gel confirmation of the Eco81I-mediated degradation of the endonuclease sensitive construct, a nanopore analysis was conducted to assess the current impedance of the resulting fragments. Due to the presence of the payload molecule, idealized current impedance predicts a larger signal for full construct when compared to the resulting products. In order to test this hypothesis, using 500 bp DNA, the scaffold:fusion:payload construct (referred to as DNA-payload below) was loaded into a nanopore with 1M LiCl, and compared before and after Eco81I-mediated degradation (FIG. 20).

In the nanopore assay, we first tested 1 nM of unconjugated 500 bp DNA alone using an 18 nm diameter pore (100 mV, 1 M LiCl). This sample produced 530 events over 32 minutes with only 7.5% exceeding a duration of 0.06 ms (FIG. 20a,b). Subsequently, DNA-payload was tested at 0.2 nM producing 117 events in 31 minutes, with 22.2% exceeding a duration of 0.06 ms (FIG. 20a,b). Next, following an incubation period between DNA-payload and Eco81I as previously described, the reaction mixture was tested on the pore at an equivalent DNA-payload concentration of 0.2 nM, producing 52 events over 40 minutes. If Eco81I cleaved a sufficient majority of the cleavable linker, the reaction mixture would contain DNA alone and payload alone, and as a result would have a reduction in the percentage of events exceeding 0.06 ms in duration compared to the un-degraded DNA-payload. This was in fact the case, with 7.7% of events exceeding 0.06 ms for DNA-payload post Eco81I degradation, a reduction from 22.2% for DNA-payload without degradation (FIG. 20a,b). A plot of Q(p)±Q_{sd}(p) as a function of recording time is shown for each reagent type (FIG. 20b).

We again implemented the method described previously for assigning statistical significance to nanopore detection assays. Specifically, with equation (2), we can implicitly detect Eco81I activity by testing for the absence of the DNA-payload molecular construct using the data generated by the reaction mixture between DNA-payload and Eco81I. In this case, DNA-payload is the type 2 molecule to be detected, and a minimum event duration of 0.06 ms is chosen as the type 2 flagging criteria. In the control experiment with DNA-payload known to be present (without Eco81I), we establish Q(p*)=0.222. Next, treating the DNA-payload post Eco81I activity as the “unknown” data, we apply equation (2). The result is 0.0769+0.0951=0.172<0.222, which means we can say that type 2 (DNA-payload) molecules are absent with 99% confidence. As stated, the absence of DNA-payload implicitly shows that Eco81I cleaved a sufficient percentage of cleavable linkers. The Eco81I endonuclease activity result applying equation (2) is displayed in FIG. 20c.

1. A method of detecting the presence or absence of a target molecule suspected to be present in a sample, comprising:
contacting the sample with a fusion molecule comprising a cleavable linker, wherein said cleavable linker is specifically cleaved in the presence of said target molecule;
loading said sample into a device comprising a nanopore, wherein said nanopore separates an interior space of the device into two volumes;
configuring the device to pass a polymer scaffold through said nanopore, wherein a first portion of said fusion molecule is bound to said polymer scaffold, wherein a second portion of said fusion molecule is bound to a payload molecule, and wherein the device comprises a sensor configured to identify objects passing through the nanopore; and
determining with the sensor whether the cleavable linker has been cleaved, thereby detecting the presence or absence of the target molecule in said sample.

2. The method of claim 1, wherein contacting the sample with said fusion molecule is performed prior to loading said sample into said device.

3. The method of claim 1, wherein loading said sample into said device is performed prior to contacting the sample with said fusion molecule.

4. The method of claim 1, wherein said fusion molecule comprises a polymer scaffold binding domain.

5. The method of claim 4, further comprising contacting the sample with a polymer scaffold.

6. The method of claim 4, further comprising binding said polymer scaffold to said polymer scaffold binding domain.

7. The method of claim 6, wherein said polymer scaffold is bound to said polymer scaffold binding domain via a covalent bond, a hydrogen bond, an ionic bond, a van der Waals force, a hydrophobic interaction, a cation-pi interaction, a planar stacking interaction, or a metallic bond.

8. The method of claim 4, wherein said polymer scaffold binding domain comprises an azide group.

9. The method of claim 4, wherein said polymer scaffold binding domain comprises a molecule selected from the group consisting of: DNA, RNA, PNA, polypeptide, a cholesterol/DNA hybrid, and a DNA/RNA hybrid.

10. The method of claim 4, wherein said polymer scaffold binding domain comprises a molecule selected from the group consisting of: a locked nucleic acid (LNA), a bridged nucleic acid (BNA), a transcription activator-like effector nuclease (TALEN), a clustered regularly interspaced short palindromic repeat (CRISPR), an aptamer, a DNA binding protein, and an antibody fragment.

11. The method of claim 10, wherein said DNA binding protein comprises a zinc finger protein.

12. The method of claim 10, wherein said antibody fragment comprises a fragment antigen-binding (Fab) fragment.

13. The method of claim 4, wherein said polymer scaffold binding domain comprises a chemical modification.

14. The method of claim 1, wherein said fusion molecule comprises a payload molecule binding domain.

15. The method of claim 14, further comprising contacting the sample with a payload molecule.

16. The method of claim 14, further comprising binding said payload molecule to said payload molecule binding domain.

17. The method of claim 16, wherein said payload molecule binds to said payload molecule binding domain via a covalent bond, a hydrogen bond, an ionic bond, a van der Waals force, a hydrophobic interaction, a cation-pi interaction, a planar stacking interaction, or a metallic bond.

18. The method of claim 14, wherein said payload molecule binding domain comprises DBCO.

19. The method of claim 1, wherein said fusion molecule comprises a polymer scaffold binding domain and a payload molecule binding domain.

20. The method of claim 1, wherein said first portion of said fusion molecule is bound directly or indirectly to said polymer scaffold via a covalent bond, a hydrogen bond, an ionic bond, a van der Waals force, a hydrophobic interaction, a cation-pi interaction, a planar stacking interaction, or a metallic bond.

21. The method of claim 1, wherein said second portion of said fusion molecule is bound directly or indirectly to said payload molecule via a covalent bond, a hydrogen bond, an ionic bond, a van der Waals force, a hydrophobic interaction, a cation-pi interaction, a planar stacking interaction, or a metallic bond.

22. The method of claim 1, wherein said payload molecule or said polymer scaffold is bound to said fusion molecule via direct covalent tethering.

23. The method of claim 22, wherein said fusion molecule comprises a connector for direct covalent tethering of said polymer scaffold or said fusion molecule to said cleavable linker.

24. The method of claim 1, wherein said polymer scaffold comprises said fusion molecule.

25. The method of claim 1, wherein said detection comprises determining with a sensor whether the polymer scaffold is bound to the payload molecule via the fusion molecule.

26. The method of claim 1, wherein said sensor detects an electrical signal in said nanopore.

27. The method of claim 26, wherein said electrical signal is an electrical current.

28. The method of claim 1, wherein said target molecule is a hydrolase or lyase.

29. The method of claim 1, wherein said cleavable linker comprises a molecule selected from the group consisting of: a deoxyribonucleic acid (DNA), a ribonucleic acid (RNA), and a polypeptide.

30. The method of claim 1, wherein said cleavable linker is selected from the group consisting of: an azo compound, a disulfide bridge, a sulfone, an ethylene glycolyl disuccinate, a hydrazone, an acetal, an imine, a vinyl ether, a vicinal diol, and a picolinate ester.

31. The method of claim 1, wherein said target molecule specifically cleaves a bond in said cleavable linker selected from the group consisting of: a carbon-oxygen bond, a carbon-sulfur bond, a carbon-nitrogen bond, and a carbon-carbon bond.

32. The method of claim 1, wherein said polymer scaffold comprises a molecule selected from the group consisting of: a deoxyribonucleic acid (DNA), a dendrimer, a peptide nucleic acid (PNA), a ribonucleic acid (RNA), a polypeptide, a nanorod, a nanotube, a cholesterol/DNA hybrid, and a DNA/RNA hybrid

33. The method of claim 1, wherein said payload molecule comprises a molecule selected from the group consisting of: a dendrimer, a double stranded DNA, a single stranded DNA, a DNA aptamer, a fluorophore, a protein, a polypeptide, a nanobead, a nanorod, a nanotube, a fullerene, a PEG molecule, a liposome, and a cholesterol-DNA hybrid.

34. The method of claim 1, wherein the sensor comprises an electrode pair, wherein said electrode pair applies a voltage differential between the two volumes and detects current flow through the nanopore.

35. The method of claim 1, wherein the fusion molecule comprises two or more cleavable linkers.

36. The method of claim 1, wherein said device comprises at least two nanopores in series, wherein said polymer scaffold is simultaneously captured and detected in said at least two nanopores.

37. The method of claim 36, wherein the translocation of said polymer scaffold is controlled by applying a unique voltage across each of said nanopores.

38. A method of detecting the presence or absence of a target molecule or condition suspected to be present in a sample, comprising:
contacting the sample with a fusion molecule comprising a cleavable linker, wherein said cleavable linker is specifically cleaved in the presence of said target molecule or condition;
loading said sample into a device comprising a nanopore, wherein said nanopore separates an interior space of the device into two volumes;
configuring the device to pass a polymer scaffold through said nanopore, wherein a first portion of said fusion molecule is bound to said polymer scaffold, wherein a second portion of said fusion molecule is bound to a payload molecule, and wherein the device comprises a sensor configured to identify objects passing through the nanopore; and
determining with the sensor whether the cleavable linker has been cleaved, thereby detecting the presence or absence of the target molecule or condition in said sample.

39. A method for detecting the presence or absence of a target molecule or condition suspected to be present in a sample, comprising:
contacting the sample with a fusion molecule, a polymer scaffold, and a payload molecule, said fusion molecule comprising a cleavable linker, wherein said target molecule specifically cleaves said cleavable linker, a polymer scaffold binding domain, and a payload molecule binding domain;
loading said fusion molecule, said polymer scaffold, said payload molecule, and said sample into a device comprising a nanopore, wherein said nanopore separates an interior space of the device into two volumes;
configuring the device to pass the polymer scaffold through said nanopore, wherein the device comprises a sensor configured to identify objects passing through the nanopore; and
determining with the sensor whether the cleavable linker is bound to the payload molecule, thereby detecting the presence or absence of the target molecule or condition.

40. The method of claim 39, wherein the target molecule comprises a hydrolase or lyase.

41. The method of claim 39, wherein the target molecule or condition photolytically cleaves the cleavable linker via exposure of said cleavable linker to light comprising a wavelength of 10 nm to 550 nm.

42. The method of claim 41, wherein the cleavable linker sensitive to photolytic cleavage is selected from the group consisting of: an ortho-nitrobenzyl derivative and a phenacyl ester derivative.

43. The method of claim 39, wherein the target molecule or condition chemically cleaves the cleavable linker via exposure of said cleavable linker to a reagent selected from the group consisting of: a nucleophilic reagent, a basic reagent, an electrophilic reagent, an acidic reagent, a reducing reagent, an oxidizing reagent, and an organometallic compound.

44. The method of claim 39, wherein at least one of said two volumes in said device comprises conditions allowing binding of said fusion molecule to said polymer scaffold and binding of said fusion molecule to said payload molecule.

45. The method of claim 39, wherein said fusion molecule is bound to said polymer scaffold and said payload molecule prior to contacting the sample with said fusion molecule.

46. The method of claim 39, wherein said fusion molecule is bound to said polymer scaffold and said payload molecule, prior to loading said fusion molecule into said device.

47. The method of claim 39, wherein one or more volumes within said device comprises conditions allowing said target molecule or said condition suspected to be present in said sample to cleave said cleavable linker.

48. The method of claim 39, wherein contacting the sample with said fusion molecule is performed prior to loading said sample into said device.

49. The method of claim 39, wherein loading said sample into said device is performed prior to contacting the sample with said fusion molecule.

50. The method of claim 39, wherein the polymer scaffold comprises a molecule selected from the group consisting of: deoxyribonucleic acid (DNA), a dendrimer, a peptide nucleic acid (PNA), a ribonucleic acid (RNA), a polypeptide, a nanorod, a nanotube, a cholesterol/DNA hybrid, and a DNA/RNA hybrid.

51. The method of claim 39, wherein the cleavable linker comprises a molecule selected from the group consisting of: a deoxyribonucleic acid (DNA), a ribonucleic acid (RNA), and a polypeptide.

52. The method of claim 39, wherein said target molecule or condition specifically cleaves a bond in said cleavable linker selected from the group consisting of: a carbon-oxygen bond, a carbon-sulfur bond, a carbon-nitrogen bond, and a carbon-carbon bond.

53. The method of claim 39, wherein said cleavable linker is selected from the group consisting of: an azo compound, a disulfide bridge, a sulfone, an ethylene glycolyl disuccinate, a hydrazone, an acetal, an imine, a vinyl ether, a vicinal diol, and a picolinate ester.

54. The method of claim 39, wherein the payload molecule comprises a molecule selected from the group consisting of: a dendrimer, a double stranded DNA, a single stranded DNA, a DNA aptamer, a fluorophore, a protein, a polypeptide, a nanobead, a nanorod, a nanotube, a fullerene, a PEG molecule, a liposome, and a cholesterol-DNA hybrid.

55. The method of claim 39, wherein said polymer scaffold and said fusion molecule are bound via a covalent bond, a hydrogen bond, an ionic bond, a van der Waals force, a hydrophobic interaction, a cation-pi interaction, a planar stacking interaction, or a metallic bond.

56. The method of claim 55, wherein said scaffold and said fusion molecule are bound via direct covalent tethering.

57. The method of claim 55, wherein said fusion molecule comprises a connector for direct covalent tethering to said polymer scaffold, wherein the connector is bound to said cleavable linker.

58. The method of claim 57, wherein said connector comprises polyethylene glycol.

59. The method of claim 55, wherein said fusion molecule comprises a polymer scaffold binding domain comprising a molecule selected from the group consisting of: DNA, RNA, PNA, polypeptide, a cholesterol/DNA hybrid, and a DNA/RNA hybrid.

60. The method of claim 55, wherein said fusion molecule comprises a molecule selected from the group consisting of: a locked nucleic acid (LNA), a bridged nucleic acid (BNA), a transcription activator-like effector nuclease (TALEN), a clustered regularly interspaced short palindromic repeat (CRISPR), an aptamer, a DNA binding protein, and an antibody fragment.

61. The method of claim 60, wherein said DNA binding protein comprises a zinc finger protein.

62. The method of claim 60, wherein said antibody fragment comprises a fragment antigen-binding (Fab) fragment.

63. The method of claim 55, wherein said fusion molecule comprises a chemical modification.

64. The method of claim 39, wherein the cleavable linker and the payload molecule are bound directly or indirectly via a covalent bond, a hydrogen bond, an ionic bond, a van der Waals force, a hydrophobic interaction, a cation-pi interaction, a planar stacking interaction, or a metallic bond.

65. The method of claim 39, wherein the sensor comprises an electrode pair, wherein said electrode pair applies a voltage differential between the two volumes and detects current flow through the nanopore.

66. The method of claim 39, wherein the fusion molecule comprises two or more cleavable linkers.

67. A method for detecting a target molecule or condition suspected to be present in a sample, comprising:
contacting the sample with a polymer scaffold, wherein the scaffold comprises a cleavable domain, wherein said cleavable domain is specifically cleaved in the presence of said target molecule;
loading said polymer scaffold and said sample into a device comprising a nanopore, wherein said nanopore separates an interior space of the device into two volumes;
configuring the device to pass the polymer scaffold through said nanopore, wherein the device comprises a sensor configured to identify objects passing through the nanopore; and
determining with the sensor whether the cleavable domain has been cleaved, thereby detecting the presence or absence of the target molecule or condition in said sample.

68. The method of claim 67, wherein the polymer scaffold comprises a molecule selected from the group consisting of: a deoxyribonucleic acid (DNA), a dendrimer, a peptide nucleic acid (PNA), a ribonucleic acid (RNA), a polypeptide, a nanorod, a nanotube, a cholesterol/DNA hybrid, and a DNA/RNA hybrid.

69. The method of claim 67, wherein the cleavable domain comprises a molecule selected from the group consisting of: a deoxyribonucleic acid (DNA), a ribonucleic acid (RNA), and a polypeptide.

70. The method of claim 67, wherein the target molecule or condition specifically cleaves a bond of said cleavable domain selected from the group consisting of: a carbon-oxygen bond, a carbon-sulfur bond, a carbon-nitrogen bond, and a carbon-carbon bond.

71. The method of claim 67, wherein the cleavable domain is photolytically cleaved in the presence of said target molecule or condition, and wherein said cleavable domain comprises a molecule selected from the group consisting of: an ortho-nitrobenzyl derivative and a phenacyl ester derivative.

72. The method of claim 67, wherein the cleavable domain is chemically cleaved in the presence of said target molecule or condition, and wherein the cleavable domain comprises a molecule selected from the group consisting of: an azo compound, a disulfide bridge, a sulfone, an ethylene glycolyl disuccinate, a hydrazone, an acetal, an imine, a vinyl ether, a vicinal diol, or a picolinate ester.

73. The method of claim 67, wherein said device comprises at least two nanopores in series, and wherein said polymer scaffold is simultaneously in said at least two nanopores during translocation.

74. A method of quantitating a target molecule or condition suspected to be present in a sample, comprising:
contacting the sample with a fusion molecule, a polymer scaffold, and a payload molecule, said fusion molecule comprising a cleavable linker, wherein said cleavable linker is specifically cleaved in the presence of said target molecule or condition, a polymer scaffold binding domain, and a payload molecule binding domain;
loading said fusion molecule, said polymer scaffold, said payload molecule, and said sample into a device comprising a nanopore, wherein said nanopore separates an interior space of the device into two volumes;
configuring the device to pass the polymer scaffold through said nanopore, wherein the device comprises a sensor configured to identify objects passing through the nanopore;
determining with the sensor whether the polymer scaffold is bound to the payload molecule, thereby detecting the presence or absence of target molecule; and
estimating the concentration or activity of the target molecule or condition suspected to be present in a sample using measurements from said sensor.

75. The method of claim 74, wherein said determination of the concentration or activity comprises assigning a numerical confidence value to detection of the target molecule or condition suspected to be present in the sample.

76. The method of claim 74, wherein said steps of contacting the sample with said fusion molecule, loading said fusion molecule, said polymer scaffold, said payload molecule, and said sample into the device, configuring the device, and determining whether the polymer scaffold is bound to the payload molecule are repeated for varying concentrations or activity of one or more of said polymer scaffold, said fusion molecule, said payload molecule or said target molecule or condition in said sample.

77. A method of quantitating a target molecule suspected to be present in a sample, comprising:
contacting the sample with a fusion molecule comprising a cleavable linker, wherein said cleavable linker is specifically cleaved in the presence of said target molecule;
loading said sample into a device comprising a nanopore, wherein said nanopore separates an interior space of the device into two volumes;
configuring the device to pass a polymer scaffold through said nanopore, wherein a first portion of said fusion molecule is bound to said polymer scaffold, wherein a second portion of said fusion molecule is bound to a payload molecule, and wherein the device comprises a sensor configured to identify objects passing through the nanopore;
determining with the sensor whether the cleavable linker has been cleaved, thereby detecting the presence or absence of the target molecule in said sample; and
estimating the concentration of the target molecule or condition suspected to be present in a sample using measurements from said sensor.

78. The method of claim 77, wherein said determination of the concentration comprises assigning a numerical confidence value to detection of the target molecule or condition suspected to be present in the sample.

79. The method of claim 77, wherein said steps of contacting the sample with said fusion molecule, loading said sample into the device, configuring the device, and determining whether the cleavable linker has been cleaved are repeated for varying concentrations of one or more of said polymer scaffold, said fusion molecule, said payload molecule or said target molecule or condition in said sample.

80. A kit comprising:
a device comprising a nanopore, wherein said nanopore separates an interior space of the device into two volumes, and configuring the device to pass the nucleic acid through one or more pores, wherein the device comprises a sensor for each pore that is configured to identify objects passing through the nanopore;
a fusion molecule comprising a cleavable linker, wherein said cleavable linker is specifically cleaved in the presence of a target molecule;
a payload molecule;
a polymer scaffold; and
instructions for use to detect the presence or absence of said target molecule in a sample.

81. The kit of claim 80, wherein said fusion molecule is bound to said payload molecule.

82. The kit of claim 80, wherein said fusion molecule is bound to said polymer scaffold.
